(19) United States
(12) Patent Application Publication
(10) Pub. No.: US 2016/0106814 A1
(43) Pub. Date: Apr. 21, 2016

(54) Novel Oral Pharmaceutical Composition for Treatment of Diabetes

(71) Applicant: NOVO NORDISK A/S, Bagsværd (DK)

(72) Inventors: Salka Elboel Rasmussen, Vaerloese (DK); Frantisek Hubalek, Herlev (DK); Andreanne Bouchard, Vaerloese (DK); Abdallah Makhlof, Bagsvaerd (DK); Betty Lomstein Pedersen, Glostrup (DK)

(21) Appl. No.: 14/893,693

(22) PCT Filed: May 30, 2014
(86) PCT No.: PCT/EP2014/061237

§ 371 Date: Nov. 24, 2015

(30) Foreign Application Priority Data
May 30, 2013 (EP) 13169897.9

(51) Int. Cl. A61K 38/26; A61K 9/00; A61K 38/16; A61K 9/48; A61K 38/28; A61K 38/56

(52) U.S. Cl. CPC A61K 38/26; A61K 38/28; A61K 38/56; A61K 38/168; A61K 9/4858; A61K 9/0053

(57) ABSTRACT

The invention provides improved solid oral pharmaceutical compositions comprising an insulin peptide or GLP-1 peptide and methods of producing such.

### TECHNICAL FIELD

The invention is related to novel oral pharmaceutical compositions comprising an insulin peptide or GLP-1 peptide and methods for producing such.

### BACKGROUND

The oral route is by far the most widely used route for drug administration. Administration of peptides and proteins is however often limited to parenteral routes rather than the preferred oral administration due to several barriers such as enzymatic degradation in the gastrointestinal (GI) tract and intestinal mucosa, as well as insufficient and variable absorption from the intestinal mucosa.

To overcome this barrier, permeation enhancers and inhibitors of proteolytic enzymes are commonly included in oral formulations.

Oral formulations for peptides and proteins comprising permeation enhancer(s) and protease inhibitor(s) have for long been proposed to enable oral absorption of pharmacologically active peptides or proteins such as insulin and GLP-1. However, it has turned out not to be trivial to develop and prepare an oral solid formulation for a drug product.

The use of the Bowman Birk (BBI) inhibitor from soy has been suggested as a useful protease inhibitor for oral administration of peptides and proteins.

A well-known permeation enhancer for oral delivery of proteins and peptides is capric acid or sodium caprate.

Kyeongsoon Park et al disclose in “Oral protein delivery: Current status and future prospect”, Reactive & Functional Polymers, vol. 71, no. 3, (2011) pages 280-287 an overview over technologies under development for oral protein delivery. WO09118722 A2 describes compositions that include a protein and at least two protease inhibitors and US 2005/232981 describes water soluble compositions immersed in a hydrophobic medium with a fluidizing agent.

However, information on how to produce a functioning oral pharmaceutical composition by combining e.g. sodium caprate with a protease inhibitor has not been provided. Thus, there is still a need for oral pharmaceutical compositions for oral delivery of peptides and proteins, which are effective in providing therapeutically effective blood levels of the therapeutically active peptide ingredient to a subject when administered to the gastrointestinal tract.

### SUMMARY

The present invention provides an improved solid oral pharmaceutical composition comprising an insulin peptide or GLP-1 peptide. Also or alternatively, the invention provides an improved solid oral pharmaceutical composition comprising an insulin peptide or a GLP-1 peptide, a salt of capric acid, Bowman-Birk Inhibitor (BBI), and a BBI solubilizing agent.

In one aspect, the invention provides an improved solid oral pharmaceutical composition comprising an insulin peptide or GLP-1 peptide, at least 10 mg salt of capric acid and at least 1 mg BBI. In one aspect, the solid oral pharmaceutical composition of the invention comprises 10 mg-400 mg BBI solubilizing agent.

In one aspect of the invention, the solubilizing agent is a sugar alcohol such as sorbitol.

In one aspect of the invention, the purity of BBI is at least 90%.

The invention may also solve further problems that will be apparent from the disclosure of the exemplary aspects.

### DESCRIPTION

The present invention is related to solid oral pharmaceutical compositions comprising a pharmaceutically active peptide or protein ingredient such as e.g. insulin or GLP-1 peptide, a salt of capric acid such as sodium caprate, and Bowman-Birk Inhibitor (BBI).

As we describe in this invention, the successful combination of BBI and a salt of capric acid is not trivial, since the permeation enhancer and the protease inhibitor interact with each other, and with the therapeutically active peptide ingredient.

In one aspect, the invention is related to a solid oral pharmaceutical composition comprising a) an active peptide ingredient which is an insulin peptide or GLP-1 peptide, b) a salt of capric acid such as sodium caprate, c) Bowman-Birk Inhibitor (BBI)), and d) a BBI solubilizing agent, such as a sugar alcohol, such as sorbitol or mannitol.

The term “capric acid” is herein used to mean a saturated fatty acid of formula CH₃(CH₂)₈COOH. Alternative names for capric acid includes: Decanoic acid, n-Capric acid, n-Decanoic acid, Decylic acid and n-Decylic acid.

In one aspect of the invention the salt of capric acid is sodium caprate.

In one aspect, the salt of capric acid is a delivery agent, i.e. an absorption enhancer useful for oral delivery of an active ingredient which is a peptide or protein.

The term “delivery agent” is herein used for biologicals or chemicals that promote the intestinal absorption of active peptide ingredients i.e. increasing permeability of poorly permeable peptide pharmaceuticals and thereby improve oral drug bioavailability. Delivery of a pharmaceutical by oral route is thus predominantly restricted by pre-systemic degradation and poor penetration across the gut wall. One of the major challenges in the oral drug delivery is the development of novel dosage forms to endorse absorption of poorly permeable drugs across the intestinal epithelium.

Bowman-Birk Inhibitors (BBIs) are known to the person skilled in the art as serine protease inhibitors. It is thus known that plants may contain a variety of serine protease inhibitors, which can be divided into different families. The Bowman-Birk inhibitors (BBIs) belong to an extensively studied family of serine protease inhibitors that are abundant in dicotyledonous and monocotyledonous plants.

BBI are named after the scientists who first isolated and characterized a member of this family from soybean (Bowman D E, Differentiation of soy bean antitryptic factor, Proc Soc Exp Biol Med 63: 547-550 (1946); Birk Y, Gertler A, Khalef S, A pure trypsin inhibitor from soya bean, Biochem J 87: 281-284 (1963)). BBI proteins have e.g. been found in leguminous plants, such as soybean, pea, lentil, peanut and chickpea and in the Poaceae family. Serine proteinase inhibitors of the BBI family interact with the enzymes they inhibit via an exposed surface loop that adopts the canonical proteinase inhibitory conformation. The resulting noncovalent complex renders the proteinase inactive. A particular feature of the Bowman-Birk inhibitor protein, however, is that the interacting loop is a particularly well-defined, disulphide-linked, short beta-strand region. Typical member of BBI family contains two such interactive loops and thus inhibiting up to two serine proteases. BBI family proteins typically consist of 50-80 amino acids, and contain seven disulfide bonds with a conserved disulfide pattern.

The terms “protease inhibitor” or “enzyme inhibitor” as used herein refer to molecules that inhibit the function of proteases. In one aspect of the invention, the protease inhibitors inhibit proteases from the class of serine proteases (serine protease inhibitors). In one aspect of the invention, the protease inhibitor inhibits pancreatic enzymes found in the gastro intestinal tract in mammals.

Pancreatic enzymes are enzymes present in the pancreatic juice and includes lipases, proteases and amylases such as e.g. trypsin, chymotrypsin, carboxypeptidase, elastase, pancreatic lipase, sterol esterase, phospholipase, various nucleases and pancreatic amylase. Protease inhibitors inhibiting pancreatic enzymes found in the gastro intestinal tract in mammals thus inhibit e.g. the enzymes trypsin, chymotrypsin, carboxypeptidase, elastase, pancreatic lipase, sterol esterase, phospholipase, various nucleases and/or pancreatic amylase.

In one aspect of the invention, protease inhibitor is a compound that binds to proteolytic enzymes in such a way to interfere with degradation of peptides/proteins.

In general, compounds can bind to proteolytic enzymes at many different sites, however, it is only binding that interferes with the function of proteolytic enzymes that is of interest when searching for inhibitors of proteolysis. The best way to look for inhibitors is to examine the effect of the presence of the potential inhibitor on the enzymatic reaction catalyzed by the protease in question. Enzyme kinetics describes several possibilities for a compound to inhibit an enzyme as known to the person skilled in the art. Enzyme inhibition may be, for example, competitive, non-competitive, mixed. Procedures for distinguishing different kinds of enzyme inhibition were previously described in many scientific articles and numerous textbooks, for example, Fundamentals of Enzyme Kinetics by Athel Cornish-Bowden ISBN-13: 978-3527330744. In addition to enzyme kinetics, interactions of proteolytic enzymes with their inhibitors are commonly examined by many different methods, for example, x-ray crystallography, NMR spectroscopy, numerous spectroscopy techniques (fluorescence, circular dischroism, UV-VIS), mass spectrometry, calorimetry, etcetera as known to the person skilled in the art. Compounds may also strongly bind to an enzyme but not affect the rate of the catalyzed reaction.

BBI may be obtained by various methods known to the person skilled in the art such as by recombinant production or by isolation from plants.

The terms “BBI” or “Bowman Birk” or “Bowman Birk inhibitor” as used interchangeably herein refer to inhibitors from the Bowman Birk family of serine protease inhibitors such as, but not limited to, the Bowman Birk inhibitor isolated from Glycine max (soybean), other leguminous plants or plants from the Poaceae family, or Bowman Birk inhibitor recombinantly expressed in cell based systems such as, but not limited to, E. Coli, Bacillus subtilis or plant based cell cultures.

In one aspect of the invention, the Bowman Birk inhibitor is isolated from plants. In one aspect, the Bowman Birk inhibitor is isolated from legumes. In one aspect, the Bowman Birk inhibitor is isolated from soybeans or soy fractions. Methods for isolation of BBI from e.g. soybeans or soy fractions are known in the art (e.g. Gladysheva, I P et al., Isolation and characterization of soybean Bowman-Birk inhibitor from different sources, Biochemistry (Mosc), 65(2):198-203 (2000); Garcia, M C et al., Composition and Characterization of Soyabean and Related Products, Critical Reviews in Food Science and Nutrition, 37(4):361-391 (1997); Yeboah, N A et al., A rapid purification method for soybean Bowman-Birk protease inhibitor using hydrophobic-interaction chromatography, Protein expression and purification 7(3):309-314 (1996), JP63051335-A, U.S. Pat. No. 4,793,996-A, WO2003007976-A and WO2011082338-A1), or purchased from commercial sources.

The purity as used herein of BBI is a function of total BBI protein concentration, specific activity (as measured by chymotrypsin inhibitor units/g protein), and the absence of components that function as antagonists for BBI, toxins, or other components that have deleterious effect beyond merely diluting the efficiency per unit quantity of the BBI. Generally, the total BBI protein concentration of BBI products of the present disclosure is at least about 90 wt. %. Typically, the total BBI protein concentration of the BBI products of the present disclosure is at least about 90 wt. %, at least about 91 wt. %, at least about 92 wt. %, at least about 93 wt. %, at least about 94 wt. %, at least about 95 wt. %, at least about 96 wt. %, at least about 97 wt. %, at least about 98 wt. %, and at least about 99 wt. %.

In one aspect BBI, when used for the invention, has a purity as represented by a total BBI protein concentration which is at least 90 wt. %, such as at least 92 wt. %, 94 wt. % or 95 wt. %. In one aspect, BBI has a purity as represented by a total BBI protein concentration which is at least 96 wt. %, In one aspect, BBI has a purity which is at least 97 wt. %, at least 98 wt. % or at least 99 wt. %.

In one aspect of the invention, total BBI protein concentration includes truncated forms of BBI. Truncated forms of BBI have the same sequence as BBI except they are missing 1-15 amino acids from their C-terminus and/or N-terminus. In one aspect of the invention, total BBI protein concentration is without truncated forms of BBI.

It will be apparent to the person skilled in the art how to measure purity of BBI for use in the invention. The purity may e.g. be measured after electrophoresis on a one- or two-dimensional SDS-PAGE gel and/or after RP-HPLC separation. One non-limiting example of how purity may be measured is e.g. illustrated in Example 2 herein.

A “pure” monomeric protein will yield a single band after electrophoresis on a one- or two-dimensional SDS-PAGE gel, will elute from a gel filtration, high performance liquid chromatography (HPLC), or ion exchange column as a single symmetrical absorbance peak, will yield a single set of mass spectrometric, nuclear magnetic resonance (NMR), or W absorbance spectral signals, and where appropriate, will be free of contaminating enzyme activities. Since absolute purity can never be established, a simple criterion of purity is used routinely, namely, the inability to detect more than a single band of protein after SDS-PAGE. (See Mohan, Determination of purity and yield. Methods in Molecular Biology, 11, 307-323 (1992)).

BBI protein content of products, i.e. the amount of BBI present in a product of the present disclosure may be determined by conventional methods known in the art including, for example, chromatographic methods (for example, Reverse Phase High pressure Liquid Chromatography (RP-HPLC), size exclusion or gel permeation HPLC, ion exchange HPLC, etcetera), and/or spectroscopic methods (for example NMR, UV-VIS, CD, IR, etcetera), and/or antibody based methods (for example ELISA, LOCI, RIA, etcetera), and/or general protein content methods (for example Bradford method, the Lowry method described in Ohnishi, S. T., and Barr, J. K., A simplified method of quantitating proteins using the biuret and phenol reagents. Anal. Biochem., 86, 193 (1978), etcetera). The results obtained by the general protein content method will reflect total amount of protein present in the products including also the active ingredient (API) if said API is of protein origin and in order to obtain BBI protein content the amount of API needs to be subtracted from the results obtained by these methods.

In one aspect of the invention, the Bowman Birk inhibitor is recombinantly expressed in a cell based system. In one aspect of the invention, the Bowman Birk inhibitor is recombinantly expressed in a cell based system such as, but not limited to, E. Coli, Bacillus subtilis or plant based cell cultures. Methods for recombinantly expression of BBI are known in the art (non-limiting examples of recombinant methods are e.g. disclosed in Li N. et al., The refolding, purification, and activity analysis of a rice Bowman-Birk inhibitor expressed in Escherichia coli. Protein Expr Purif. 1999 February; 15(1):99-104 or Vogtentanz G et al., Protein Expr Purif. 2007 September; 55(1):40-52).

In one aspect of the invention, the solid oral pharmaceutical composition comprises at least 10 mg salt of capric acid. In one aspect of the invention, the solid oral pharmaceutical composition comprises up to 450 mg salt of capric acid. In one aspect of the invention, the solid oral pharmaceutical composition comprises up to 275 mg salt of capric acid. In one aspect of the invention, the solid oral pharmaceutical composition comprises up to 200 mg salt of capric acid. In one aspect of the invention the solid oral pharmaceutical composition comprises about 275 mg capric acid. In one aspect of the invention, the solid oral pharmaceutical composition comprises between 10 mg and 450 mg salt of capric acid, in one aspect between 10 mg and 350 mg salt of capric acid, in one aspect between 10 mg and 275 mg salt of capric acid, and in one aspect between 10 mg and 200 mg salt of capric acid.

In one aspect of the invention, the solid oral pharmaceutical composition comprises at least 1 mg BBI. In one aspect the solid oral pharmaceutical composition comprises about 10 mg BBI, in one aspect about 25 mg BBI, in one aspect about 50 mg BBI and in one aspect the solid oral pharmaceutical composition comprises about 200 mg BBI. In one aspect the solid oral pharmaceutical composition comprises between 1 mg and 200 mg BBI, in one aspect between 10 mg and 200 mg BBI, in one aspect between 25 mg and 200 mg BBI and in one aspect the solid oral pharmaceutical composition comprises between 50 mg and 200 mg BBI. In one aspect the solid oral pharmaceutical composition comprises between 1 mg and 50 mg BBI, in one aspect between 1 mg and 25 mg BBI and in one aspect the solid oral pharmaceutical composition comprises between 1 mg and 10 mg BBI. In one aspect the solid oral pharmaceutical composition comprises between 10 mg and 50 mg BBI, in one aspect between 10 mg and 25 mg BBI and in one aspect the solid oral pharmaceutical composition comprises between 25 mg and 50 mg BBI.

The invention also covers the use of a BBI solubilizing agent, such as e.g. a sugar alcohol in the solid oral pharmaceutical composition. The inventors have thus surprisingly found that the addition of a BBI solubilizing agent may enhance the in vivo effect.

The term “BBI solubilizing agent” is herein used to mean an agent which promotes BBI dissolution in vivo. The agent thus enhances the bioavailability of the active peptide ingredient within the body by promoting more effective drug release.

In one aspect of the invention the BBI solubilizing agent is a sugar alcohol. In one aspect, the BBI solubilizing agent is sorbitol or mannitol.

When used herein the term “sugar alcohol” means a hydrogenated form of a carbohydrate having the general formula H(HCHO)ₙ₊₁H. A sugar alcohol is thus a carbohydrate whose carbonyl group has been reduced to a primary or secondary hydroxyl group. Exemplary sugar alcohols are e.g. sorbitol and mannitol.

Any suitable sugar alcohol may be included in the solid oral pharmaceutical composition of the present invention. As used herein, the “sugar alcohols” used in the invention include monosaccharides, di-saccharides and oligosaccharides. Exemplary sugar alcohols include, but are not limited to, xylitol, mannitol, sorbitol, erythritol, lactitol, pentitol and hexitol. Exemplary monosaccharides include, but are not limited to, glucose, fructose, aldose and ketose. Exemplary di-saccharides include, but are not limited to, sucrose, isomalt, lactose, trehalose, and maltose. Exemplary oligosaccharides include, but are not limited to, maltotriose, raffinose and maltotetraose. In one aspect, the sugar alcohol is sorbitol, mannitol or xylitol. In one aspect, the sugar alcohol is sorbitol. In one aspect, the sugar alcohol is a disaccharide. In one aspect, the sugar alcohol is sucrose.

In one aspect of the invention, the solid oral pharmaceutical composition comprises at least 10 mg BBI solubilizing agent. In one aspect of the invention, the solid oral pharmaceutical composition comprises about 275 mg BBI solubilizing agent. In one aspect of the invention, the solid oral pharmaceutical composition comprises up to 400 mg BBI solubilizing agent. In one aspect of the invention, the solid oral pharmaceutical composition comprises between 10 mg and 400 mg BBI solubilizing agent and in one aspect between 10 mg and 275 mg BBI solubilizing agent. In one aspect of the invention, the solid oral pharmaceutical composition comprises between 275 mg and 400 mg BBI solubilizing agent.

In one aspect, the solid oral pharmaceutical composition of the invention is comprised in a dosage form. In one aspect, the dosage form comprising the solid oral pharmaceutical composition of the invention is a capsule.

A “dosage form” is herein understood to mean the physical form in which a drug is produced and dispensed, such as e.g. tablet, particulate, multi-particulate, capsule, pellet, mini-tablets, encapsulated pellet, encapsulated mini-tablets, encapsulated micro-particulate, granule or mucoadhesive forms (e.g. tablets or capsules).

In one aspect, the dosage form comprising the solid oral pharmaceutical composition of the invention is a tablet.

In one aspect of the invention, the dosage form comprising the solid oral pharmaceutical composition of the invention comprises a hydroxypropyl methyl cellulose (HPMC) capsule with an enteric coat.

In one aspect of the invention, the ratio (w/w) of the salt of capric acid to BBI is between 300:1 to 1:1.

In one aspect of the invention, the ratio (w/w) of the salt of capric acid to BBI is between 45:1 to 1:1, between 30:1 to 1:1, between 9:1 to 1:1 or between 5.5:1 to 1:1.

In one aspect of the invention, the ratio (w/w) of the salt of capric acid to BBI is about 45:1, about 30:1, about 9:1, about 5.5:1, or about 1:1.

In one aspect of the invention, the ratio (w/w) of the salt of capric acid to BBI is about 5.5:1.

### Solid Oral Pharmaceutical Compositions

Solid oral pharmaceutical compositions of the invention may include encapsulation of the active peptide ingredient into nanoparticles, microparticles, granules, pellets or other kinds of multiparticulate dosage forms. Above mentioned oral pharmaceutical compositions systems may be formulated into a tablet or filled into a suitable hard-shelled or soft-shelled capsule which may be coated to release the active peptide ingredient in a controlled manner or at a preferred intestinal segment.

In one aspect the solid oral pharmaceutical composition comprises granules. In one aspect the term “granulate” refers to one or more types of granules. In one aspect the term “granule” refers to particles gathered into larger particles.

In one aspect the solid oral pharmaceutical composition is in the form of a solid dosage form. In one aspect the solid oral pharmaceutical composition is in the form of a tablet. In one aspect the solid oral pharmaceutical composition is in the form of a capsule. In one aspect the solid oral pharmaceutical composition is in the form of a sachet.

In one aspect the solid oral pharmaceutical composition comprises at least one pharmaceutically acceptable excipient.

The term “excipient” as used herein broadly refers to any component other than the active peptide ingredient(s), i.e. other than the insulin peptide or the GLP-1 peptide. The excipient may be an inert substance, which is inert in the sense that it substantially does not have any therapeutic and/or prophylactic effect per se. The excipient may serve various purposes, e.g. as a delivery agent, absorption enhancer, vehicle, solubilizing agent, filler (also known as diluents), binder, lubricant, glidant, disintegrant, crystallization retarders, acidifying agent, alkalizing agent, antioxidant, buffering agent, chelating agent, complexing agents, surfactant agent, emulsifying and/or solubilizing agents, wetting agents stabilizing agent, colouring agent, flavouring agent, and/or to improve administration, and/or absorption of the active peptide ingredient. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In one aspect the solid oral pharmaceutical composition comprises a binder. In one aspect the solid oral pharmaceutical composition comprises a disintegrant. In one aspect the solid oral pharmaceutical composition comprises a lubricant. In one aspect the solid oral pharmaceutical composition comprises one or more excipients selected from crystallization retarders, solubilizing agents (also known as surfactants), wetting agents, colouring agents, and/or pH control agents.

In one aspect the capsule including the solid oral pharmaceutical composition of the invention is size 4 to size 000 capsules such as in the range of capsule size 1 to 00, where the size is measured according to standard size definition of two-piece capsules.

The pharmaceutical composition according to the present invention may be in a dosage form of a tablet, particulate, multi-particulate, capsule, pellet, mini-tablets, encapsulated pellet, encapsulated mini-tablets, encapsulated micro-particulate, or mucoadhesive forms (e.g., tablets or capsules).

In one aspect, the pharmaceutical composition may be in a dosage form (e.g., capsule or tablet) without a coating. In one aspect, the pharmaceutical composition is in a delayed release dosage form which minimizes the release of the active peptide ingredient and the enhancer in the stomach, and hence the dilution of the local enhancer concentration therein, and releases the drug and enhancer in the intestine. In other aspects, the pharmaceutical composition is in a delayed release rapid onset dosage form. Such a dosage form minimizes the release of the active peptide ingredient and enhancer in the stomach, and hence the dilution of the local enhancer concentration therein, but releases the active peptide ingredient and enhancer rapidly once the appropriate site in the intestine has been reached, maximizing the delivery of the poorly permeable active peptide ingredient by maximizing the local concentration of the active peptide ingredient and enhancer at the site of absorption.

In one aspect, the pharmaceutical composition of the present invention may be in a form of a capsule oral dosage form. In one aspect, the capsule dosage form is an enteric coated capsule dosage form. In one aspect, the capsules dosage form is a capsule with enteric properties.

The term “capsule” as used herein includes, but is not limited to a relatively stable shell used for encapsulation of pharmaceutical formulations for oral administration. The two main types of capsules are hard-shelled capsules, which are normally used for dry, powdered ingredients, miniature pellets or mini tablets, and soft-shelled capsules, primarily used for oils and for active ingredients that are dissolved or suspended in oil. Both hard-shelled and soft-shelled capsules may be made from aqueous solutions of gelling agents such as animal protein, e.g. gelatin, or plant polysaccharides or their derivatives, e.g. carrageenans, and modified forms of starch and cellulose. Other ingredients may be added to the gelling agent solution such as plasticizers, e.g. glycerin and/or sorbitol, to decrease the capsule's hardness, coloring agents, preservatives, disintegrants, lubricants and surface treating agents.

### Methods of Preparation of Solid Oral Pharmaceutical Compositions

The solid oral pharmaceutical composition of the invention may be prepared as is known in the art. In one aspect the solid oral pharmaceutical composition may be prepared as described in the examples herein.

In one aspect the solid oral pharmaceutical composition is in the form of a capsule.

In one aspect the invention relates to a process for the preparation of a hard-shelled capsule comprising a powder or granulate comprising i) up to 15% (w/w) insulin or GLP-1 peptide, and ii) at least 15% (w/w) salt of capric acid, said method comprising the step of filling said hard-shelled capsules.

In one aspect two or more ingredients of the composition are blended. To prepare a dry blend of filling material, the various components are weighed, optionally delumped and then combined. The mixing of the components may be carried out until a homogeneous blend is obtained.

In one aspect at least a part of the composition is dry granulated or wet granulated. A granulate may be produced in a manner known to a person skilled in the art, for example by dry granulation techniques in which the pharmaceutically active ingredient and/or delivery agents are compacted with the excipients to form relatively large moldings, for example slugs or ribbons, which are comminuted by grinding, and the ground material serves as the filling material to be later filled into capsules. Suitable equipment for dry granulation includes, but is not limited to, roller compaction equipment from Gerteis, such as Gerteis MINI-PACTOR (as sold by Gerteis in 2013). In one aspect the granulate is prepared by roller compaction. In one aspect the moldings from the roller compactions process are comminuted into granules. In one aspect the term “roller compaction force” means the force between the rolls of the roller compactor when compacting materials into a continuous strip of compressed material as determined by a pressure transducer that converts the hydraulic pressure into electrical signal; the roller compaction force may be measured in kiloNewton (kN) or in kiloNewton per roll width (kN/cm).

Alternatively, a granulate may be obtained by wet granulation which may be carried out by mixing the pharmaceutically active peptide ingredient dissolved in water with a dry blend of the delivery agents and optionally one or more excipients followed by drying of the granulate.

To fill the filling material into a solid oral dosage form, for example a hard-shelled capsule, a filling equipment may be used. In a filling equipment, the filling material is filled (e.g. force fed or gravity fed) into a cavity. The filling material may or may not be compressed by the filling equipment. Subsequently, the resulting hard-shelled capsule may or may not be banded or sealed.

### Physical Properties and In Vitro Methods

It will be appreciated that the physical properties of solid oral pharmaceutical compositions of the invention should be such that the solid oral pharmaceutical compositions may be easily handled, and yet contain a minimal mass of excipients so that small, easily swallowed dosage forms which enhance patient acceptance and compliance with a high content of active peptide ingredient may be prepared, and provide dosage forms with excellent wetting, disintegration, dissolution, and ultimately, rapid and complete drug release properties.

The skilled person will be aware that a number of different parameters influence dissolution time of a solid oral pharmaceutical composition including: Dosage form (e.g. capsule or tablet), identity of active peptide ingredient, identity of additional ingredients, e.g. delivery agent, disintegrant, glidant, lubricant, diluent, amounts (ratios) of the ingredients, particle sizes and tablet hardness.

By “dissolution time” is to be understood, the time which is required for a given amount (or fraction) of a drug to be released into solution from a solid dosage form. Dissolution time is measured in vitro, under conditions that simulate those that occur in vivo, in experiments in which the amount of drug in solution is determined as a function of time.

According to an aspect, the solid oral pharmaceutical compositions of the invention have a high oral bioavailability.

Generally, the term bioavailability refers to the fraction of an administered dose of the active pharmaceutical ingredient (API), such as an insulin peptide or a GLP-1 comprised in a solid oral pharmaceutical composition of the invention that reaches the systemic circulation unchanged. By definition, when an API is administered intravenously, its bioavailability is 100%. However, when it is administered via other routes (such as orally), its bioavailability decreases (due to degradation and/or incomplete absorption and first-pass metabolism). Knowledge about bioavailability is important when calculating dosages for non-intravenous routes of administration.

A plasma concentration versus time plot is made after both oral and intravenous administration. The absolute bioavailability (F) is the area under the curve (AUC) obtained after oral administration divided by dose, divided by AUC-obtained after intravenous administration divided by dose.

In one aspect, a solid oral pharmaceutical composition of the invention has an absolute oral bioavailability when measured in dog which is at least 2%, such as at least 3%, at least 4%, or at least 5%.

Oral bioavailability and absorption kinetics of the solid oral pharmaceutical composition of the invention may be determined according to Assay (II) as described herein.

In one aspect, the solid oral pharmaceutical composition of the invention inhibits at least 1 mg of chymotrypsin. In one aspect, the solid oral pharmaceutical composition of the invention inhibits at least 2 mg of chymotrypsin. In one aspect, the solid oral pharmaceutical composition of the invention inhibits at least 3 mg of chymotrypsin. In one aspect, the solid oral pharmaceutical composition of the invention inhibits at least 5 mg of chymotrypsin. In one aspect, the solid oral pharmaceutical composition of the invention inhibits at least 10 mg of chymotrypsin. In one aspect, the solid oral pharmaceutical composition of the invention inhibits at least 20 mg of chymotrypsin. In one aspect, the solid oral pharmaceutical composition of the invention inhibits at least 30 mg of chymotrypsin. In one aspect, the solid oral pharmaceutical composition of the invention inhibits at least 50 mg of chymotrypsin. In one aspect, the solid oral pharmaceutical composition of the invention inhibits at least 100 mg of chymotrypsin. In one aspect, the solid oral pharmaceutical composition of the invention inhibits at least 150 mg of chymotrypsin.

In one aspect, the solid oral pharmaceutical composition of the invention inhibits at least 1 mg of trypsin. In one aspect, the solid oral pharmaceutical composition of the invention inhibits at least 2 mg of trypsin. In one aspect, the solid oral pharmaceutical composition of the invention inhibits at least 3 mg of trypsin. In one aspect, the solid oral pharmaceutical composition of the invention inhibits at least 5 mg of trypsin. In one aspect, the solid oral pharmaceutical composition of the invention inhibits at least 10 mg of trypsin. In one aspect, the solid oral pharmaceutical composition of the invention inhibits at least 20 mg of trypsin. In one aspect, the solid oral pharmaceutical composition of the invention inhibits at least 30 mg of trypsin. In one aspect, the solid oral pharmaceutical composition of the invention inhibits at least 50 mg of trypsin. In one aspect, the solid oral pharmaceutical composition of the invention inhibits at least 100 mg of trypsin. In one aspect, the solid oral pharmaceutical composition of the invention inhibits at least 150 mg of trypsin.

In one aspect, a solid oral pharmaceutical composition of the invention increases half-life of insulin peptide in the GI tract at least 2-fold compared to a solid oral pharmaceutical composition with said insulin peptide but without BBI and sodium caprate. In one aspect, a solid oral pharmaceutical composition of the invention increases half-life of insulin peptide in the GI tract at least 3-fold. In one aspect, a solid oral pharmaceutical composition of the invention increases half-life of insulin peptide in the GI tract at least 5-fold. In one aspect, a solid oral pharmaceutical composition of the invention increases half-life of insulin peptide in the GI tract at least 10-fold.

In one aspect, a solid oral pharmaceutical composition of the invention increases half-life of GLP-1 peptide in the GI tract at least 2-fold compared to a solid oral pharmaceutical composition with said GLP-1 peptide but without BBI and sodium caprate. In one aspect, a solid oral pharmaceutical composition of the invention increases half-life of GLP-1 peptide in the GI tract at least 3-fold. In one aspect, a solid oral pharmaceutical composition of the invention increases half-life of GLP-1 peptide in the GI tract at least 5-fold. In one aspect, a solid oral pharmaceutical composition of the invention increases half-life of GLP-1 peptide in the GI tract at least 10-fold.

Assay (I): Dissolution Test

The dissolution test is e.g. conducted with apparatus 1 (as specified in United States Pharmacopeia (USP) General Chapter <711>) using a basket rotation speed of 100 rpm. The 100 mL dissolution medium of phosphate buffer (pH 6.8) may be used at a temperature of 37° C. Enteric coated formulations are tested in the two step method. The first step is 1 h at acid pH resembling the stomach and then 2 h at a neutral pH simulating the intestines. The neutral pH may be pH 6.0, 6.5, 6.8, 7.2 or 7.4. The dissolution media may have a content of 0.1% Tween80. Sample aliquots are removed at appropriate intervals. Release is e.g. determined using a RP-HPLC method. The content is calculated based on the peak area of e.g. the insulin peptide or GLP peptide peak in the chromatogram relative to the peak areas of the insulin peptide or GLP-1 peptide reference. The HPLC method may be based on gradient elution on a C18 column. The solvent system may be trifluoroacetic acid and acetonitrile with UV detection at 215 nm.

Assay (II): Oral Administration to Beagle Dogs

Animals, Dosing and Blood Sampling:

Beagle dogs, weighing 6-17 kg during the study period are included in the study. The dogs are dosed in fasting state. The solid oral pharmaceutical compositions are administered by a single oral dosing to the dogs in groups of 8 dogs. Blood samples may be taken at the following time points: predose, 0.25, 0.5, 0.75, 1, 1.5, 2, 2.5, 3, 4, 6, 8, 10, 24, 48, 72, 96, 120, 144, 168, 192, 216, 240, 264 and 288 hours post dosing. The i.v. solution (e.g. 20 nmol/mL in a pH 7.4 solution comprising 0.1 mg/ml Tween 20, 5.5 mg/ml Phenol, 1.42 mg/ml Na2HPO4 and 14 mg/ml Propylene Glycol) is dosed in a dose volume of 0.1 mL/kg in the same dog colony in one dosing group (n=8). Blood samples may be taken at the following time points: predose, 0.083, 0.167, 0.25, 0.5, 0.75, 1, 1.5, 2, 2.5, 3, 4, 6, 8, 10, 24, 48, 72, 96, 120, 144, 168, 192, 216, 240, 264 and 288 hours post dosing.

Preparation of Plasma:

All blood samples are collected into test tubes containing EDTA for stabilisation and kept on ice until centrifugation. Plasma is separated from whole blood by centrifugation and the plasma is stored at −20° C. or lower until analysis.

Analysis of Plasma Samples:

The plasma is analysed for insulin peptide or GLP-1 peptide using a Luminescence Oxygen Channeling Immunoassay (LOCI). The LOCI assay employs donor beads coated with streptavidin and acceptor beads conjugated with a monoclonal antibody binding to a mid-molecular region of insulin peptide or GLP-1 peptide. The other monoclonal antibody, specific for an N-terminal epitope, is biotinylated. In the assay the three reactants are combined with the insulin peptide or GLP-1 peptide which form a two-sited immuno-complex. Illumination of the complex releases singlet oxygen atoms from the donor beads which channels into the acceptor beads and trigger chemiluminescence which is measured in the EnVision plate reader. The amount of light is proportional to the concentration of insulin peptide or GLP-1 peptide and the lower limit of quantification (LLOQ) in plasma is 100 pM. Alternatively, LC-MS method is used to determine concentrations of active peptide ingredients in plasma.

Assay (III): Enzyme Inhibition

The use of chromogenic substrates to monitor activity of proteolytic enzymes is known in the field (for example DelMar, E. G., et al., Anal. Biochem., 99, 316-320, (1979)). For example, N-succinyl-Ala-Ala-Pro-Phe-p-Nitroanilide is commonly used substrate for measuring chymotrypsin activity. Enzymatic cleavage of 4-nitroanilide substrates yields 4-nitroaniline (yellow color under alkaline conditions).

An assay following the increase in absorbance at 395 nm as a function of time is established in 96 well format using Varioskan Flash Multimode Meter (Thermo Scientific). Each well contains 70 μl of Dulbecco's phosphate buffer saline (Invitrogen catalogue #14190-094), 10 μl of N-succinyl-Ala-Ala-Pro-Phe-p-Nitroanilide (Sigma cat# S 7388) in dimethyl sulfoxide (DMSO), different concentrations are used in order to obtain the inhibition constant, 10 μl of sample containing BBI (for example dissolved solid dosage form, BBI solution etc) in varying concentration and 10 μl of a stock solution of chymotrypsin. The incubations are performed at 37° C. Absorbance at 395 nm is measured immediately after addition of the enzyme to the 96 well plate and also every minute for the next 80 minutes. The concentration of the enzyme is optimized to allow determination of slopes for the time course of initial absorbance increase with and without added inhibitors. The slopes are determined by linear regression of the linear part of the fluorescence trace (for example, the first 10 min of the reaction). Each assay is performed in duplicate and average of the two traces is included in the calculations. The inhibition effect may be expressed as the concentration of the sample at which the slope of the absorbance trace equals to 50% of uninhibited reaction (EC50). This is done by plotting the slopes achieved with different concentrations of the sample as a function of their concentrations and fitting the experimental results using, for example, sigmoidal logistic regression (2 parameters, Sigma Plot v 11). Inhibition constants for the interaction between BBI of the invention and proteolytic enzymes may also be obtained by performing the assay described above with varying concentrations of the inhibitor and substrate and analyzing the results, for example, by double reciprocal transformation as known to the person skilled in the art and described for example in Hubalek, F. et al J. Med. Chem. 47, 1760-1766 (2004). When replacing N-succinyl-Ala-Ala-Pro-Phe-p-Nitroanilide with N-(p-Tosyl)-Gly-Pro-Arg-p-Nitroanilide (Sigma T1637) inhibition of trypsin is measured, and when replacing with N-succinyl-Ala-Ala-Ala-p-Nitroanilide (Sigma S4760) inhibition of elastase is measured.

### Encapsulation

The solid oral pharmaceutical composition of the invention may be encapsulated. The solid oral pharmaceutical composition may be encapsulated with any available hard or soft capsule technology.

When used herein the term “hard capsule” respectively “soft capsule” in connection with capsule technology is used to mean hard-shelled capsule technology respectively soft-shelled capsule technology.

In one aspect the capsule material for a solid oral pharmaceutical composition according to present invention is hydroxypropyl methyl cellulose (HPMC). The hard capsules may also be made of gelatine or other materials suited for pharmaceutical capsule production. In one aspect the term “enteric hard” or “enteric soft” when used for capsule technology refers to hard or soft capsule technology comprising at least one element with enteric properties, such as at least one layer of an enteric coating.

The solid oral pharmaceutical composition of the invention may comprise one or more enteric or modified release coatings. The solid oral pharmaceutical composition may comprise one or more enteric or modified release coatings in addition to hard or soft capsule technology. In one aspect the enteric or modified release coating comprises at least one release modifying polymer which may be used to control the site where the insulin peptide or GLP-1 peptide ingredient is released. In one aspect the term “enteric coating” as used herein means a polymer coating that controls dissolution and release of the oral dosage form; the site of dissolution and release of the solid dosage form may be designed depending on the pH of the targeted area, where absorption of the insulin peptide or GLP-1 peptide ingredient is desired, thus also includes acid resistant protective coatings; the term includes known enteric coatings, but also any other coating with enteric properties, wherein said term “enteric properties” means properties controlling the dissolution and release of the solid oral dosage form (i.e. the solid oral pharmaceutical composition according to this invention). In one aspect the term “modified release coating” as used herein refers to a coating which comprises special excipients (e.g. a polymer) or which is prepared by special procedures, or both, designed to modify the rate, the place or the time at which the active peptide ingredient(s) are released. In one aspect modified release coating include prolonged-release coating, delayed-release coating, and pulsatile-release coating. Modified release may be achieved by pH-dependent or pH-independent polymer coating.

In one aspect the invention the capsules are coated with poly(methacrylic acid-co-ethyl acrylate (brand name: Eudragit L30D55®, as sold by Evonik Industries AG in 2013)

Coatings, such as enteric coatings, or modified release coatings may be prepared according to methods well known in the art.

### Active Peptide Ingredient

As used herein, the term “therapeutically active ingredient,” which is interchangeably used with “active ingredient” and “pharmaceutically active ingredient”, refers to any chemical compound, complex or composition that has a beneficial biological effect, preferably a therapeutic effect in the treatment of a disease or abnormal physiological condition. The terms also encompass pharmaceutically acceptable, pharmacologically active derivatives of those active agents specifically mentioned herein, including, but not limited to, salts, esters, amides, prodrugs, active metabolites, isomers, fragments, analogs, and the like. When the terms “therapeutically active ingredient” or “active ingredient” is used and when a particular active agent is specifically identified, it is to be understood that applicants intend to include the active agent per se as well as pharmaceutically acceptable, pharmacologically active salts, esters, amides, prodrugs, active metabolites, isomers, fragments, analogs, etc.

The therapeutically active ingredient of the present invention includes any active ingredient that is appropriate for administration via the oral route to an animal including a human.

The term “active peptide ingredient” is herein used for any drug substance in a pharmaceutical drug which is in the form of a peptide or protein and is biologically active, i.e. a peptide or a protein that provides pharmacological activity or other direct effect in the cure, treatment, or prevention of disease, or to affect the structure or any function of the body of man or animals. Alternative terms include peptide active pharmaceutical ingredient (API) and peptide bulk active.

In one aspect of the invention, the active peptide ingredient is a peptide or protein.

In one aspect of the invention, the active peptide ingredient is selected from an insulin peptide and a GLP-1 peptide.

In one aspect of the invention, the active peptide ingredient is a GLP-1 peptide.

The term “GLP-1 peptide” as used herein means a peptide which is either human GLP-1 or an analog or a derivative thereof with GLP-1 activity.

The term “human GLP-1” or “native GLP-1” as used herein means the human GLP-1 hormone whose structure and properties are well-known. Human GLP-1 is also denoted GLP-1(7-37), it has 31 amino acids and is the result from selective cleavage of the proglucagon molecule.

The GLP-1 peptides of the invention have GLP-1 activity. This term refers to the ability to bind to the GLP-1 receptor and initiate a signal transduction pathway resulting in insulinotropic action or other physiological effects as is known in the art. For example, the analogues and derivatives of the invention can be tested for GLP-1 activity using a standard GLP-1 activity assay.

The term “GLP-1 analogue” as used herein means a modified human GLP-1 wherein one or more amino acid residues of human GLP-1 have been substituted by other amino acid residues and/or wherein one or more amino acid residues have been deleted from human GLP-1 and/or wherein one or more amino acid residues have been added and/or inserted to human GLP-1.

In one aspect a GLP-1 analogue comprises 10 amino acid modifications (substitutions, deletions, additions (including insertions) and any combination thereof) or less relative to human GLP-1, alternatively 9, 8, 7, 6, 5, 4, 3 or 2 modifications or less, yet alternatively 1 modification relative to human GLP-1.

Modifications in the GLP-1 molecule are denoted stating the position, and the one or three letter code for the amino acid residue substituting the native amino acid residue.

When using sequence listing, the first amino acid residue of a sequence is assigned no. 1. However, in what follows—according to established practice in the art for GLP-1 peptides—this first residue is referred to as no. 7, and subsequent amino acid residues are numbered accordingly, ending with no. 37. Therefore, generally, any reference herein to an amino acid residue number or a position number of the GLP-1(7-37) sequence is to the sequence starting with His at position 7 and ending with Gly at position 37. Using the one letter codes for amino acids, terms like 34E, 34Q, or 34R designates that the amino acid in the position 34 is E, Q and R, respectively. Using the three letter codes for amino acids, the corresponding expressions are 34Glu, 34Gln and 34Arg, respectively.

By “des7” or “(or Des⁷)” is meant a native GLP-1 lacking the N-terminal amino acid, histidine. Thus, e.g., des7GLP-1(7-37) is an analogue of human GLP-1 where the amino acid in position 7 is deleted. This analogue may also be designated GLP-1(8-37). Similarly, (des7+des8); (des7, des8); (des7-8); or (Des⁷, Des⁸) in relation to an analogue of GLP-1(7-37), where the reference to GLP-1(7-37) may be implied, refers to an analogue in which the amino acids corresponding to the two N-terminal amino acids of native GLP-1, histidine and alanine, have been deleted. This analogue may also be designated GLP-1(9-37).

A non-limiting example of an analogue of the invention is [Aib⁸,Arg³⁴]GLP-1(7-37), which designates a GLP-1(7-37) analogue, in which the alanine at position 8 has been substituted with α-aminoisobutyric acid (Aib) and the lysine at position 34 has been substituted with arginine. This analogue may also be designated (8Aib, R34) GLP-1(7-37).

The term “GLP-1 derivative” as used herein means a chemically modified parent GLP-1(7-37) or an analogue thereof, wherein the modification(s) are in the form of attachment of amides, carbohydrates, alkyl groups, acyl groups, esters, PEGylations, combinations thereof, and the like.

In one aspect of the invention, the modification(s) include attachment of a side chain to GLP-1(7-37) or an analogue thereof. In a particular aspect, the side chain is capable of forming non-covalent aggregates with albumin, thereby promoting the circulation of the derivative with the blood stream, and also having the effect of protracting the time of action of the derivative, due to the fact that the aggregate of the GLP-1-derivative and albumin is only slowly disintegrated to release the active peptide ingredient. Thus, the substituent, or side chain, as a whole is preferably referred to as an albumin binding moiety. In particular aspects, the side chain has at least 10 carbon atoms, or at least 12, 14, 16, 18, 20, 22, or at least 24 carbon atoms. In further particular aspects, the side chain may further include at least 5 hetero atoms, in particular O and N, for example at least 7, 9, 10, 12, 15, 17, or at least 20 hetero atoms, such as at least 1, 2, or 3 N-atoms, and/or at least 3, 6, 9, 12, or 15 O-atoms.

In another particular aspect the albumin binding moiety comprises a portion which is particularly relevant for the albumin binding and thereby the protraction, which portion may accordingly be referred to as a “protracting moiety”. The protracting moiety may be at, or near, the opposite end of the albumin binding moiety, relative to its point of attachment to the peptide.

In a still further particular aspect the albumin binding moiety comprises a portion in between the protracting moiety and the point of attachment to the peptide, which portion may be referred to as a “linker”, “linker moiety”, “spacer”, or the like. The linker may be optional, and hence in that case the albumin binding moiety may be identical to the protracting moiety.

In particular aspects, the albumin binding moiety and/or the protracting moiety is lipophilic, and/or negatively charged at physiological pH (7.4).

The albumin binding moiety, the protracting moiety, or the linker may e.g. be covalently attached to a lysine residue of the GLP-1 peptide by acylation. In a preferred aspect, an active ester of the albumin binding moiety, preferably comprising a protracting moiety and a linker, is covalently linked to an amino group of a lysine residue, preferably the epsilon amino group thereof, under formation of an amide bond (this process being referred to as acylation).

Unless otherwise stated, when reference is made to an acylation of a lysine residue, it is understood to be to the epsilon-amino group thereof.

For the present purposes, the terms “albumin binding moiety”, “protracting moiety”, and “linker” may include the unreacted as well as the reacted forms of these molecules. Whether or not one or the other form is meant is clear from the context in which the term is used.

For the attachment to the GLP-1 peptide, the acid group of the fatty acid, or one of the acid groups of the fatty diacid, forms an amide bond with the epsilon amino group of a lysine residue in the GLP-1 peptide, preferably via a linker.

The term “fatty diacid” refers to fatty acids as defined above but with an additional carboxylic acid group in the omega position. Thus, fatty diacids are dicarboxylic acids.

Each of the two linkers of the derivative of the invention may comprise the following first linker element:

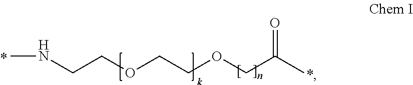

wherein k is an integer in the range of 1-5, and n is an integer in the range of 1-5.

In a particular aspect, when k=1 and n=1, this linker element may be designated OEG, or a di-radical of 8-amino-3,6-dioxaoctanic acid, and/or it may be represented by the following formula:

NH—(CH₂)₂—O—(CH₂)₂—O—CH₂—CO—*. Chem II:

In another particular aspect, each linker of the derivative of the invention may further comprise, independently, a second linker element, preferably a Glu di-radical, such as Chem III and/or Chem IV:

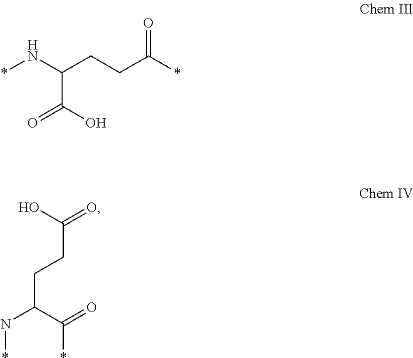 [SMILES: *NC(CCC(*)=O)C(=O)O ; *NC(CCC(=O)O)C(*)=O]

wherein the Glu di-radical may be included p times, where p is an integer in the range of 1-3.

Chem III may also be referred to as gamma-Glu, or briefly γGlu, due to the fact that it is the gamma carboxy group of the amino acid glutamic acid which is here used for connection to another linker element, or to the epsilon-amino group of lysine. As explained above, the other linker element may, for example, be another Glu residue, or an OEG molecule. The amino group of Glu in turn forms an amide bond with the carboxy group of the protracting moiety, or with the carboxy group of, e.g., an OEG molecule, if present, or with the gamma-carboxy group of, e.g., another Glu, if present.

Chem IV may also be referred to as alpha-Glu, or briefly aGlu, or simply Glu, due to the fact that it is the alpha carboxy group of the amino acid glutamic acid which is here used for connection to another linker element, or to the epsilon-amino group of lysine.

The above structures of Chem. III and Chem. IV cover the L-form, as well as the D-form of Glu. In particular aspects, Chem. III and/or Chem. IV is/are, independently, a) in the L-form, or b) in the D-form.

In still further particular aspects the linker has a) from 5 to 41 C-atoms; and/or b) from 4 to 28 hetero atoms.

The concentration in plasma of the GLP-1 derivatives of the invention may be determined using any suitable method. For example, LC-MS (Liquid Chromatography Mass Spectroscopy) may be used, or immunoassays such as RIA (Radio Immuno Assay), ELISA (Enzyme-Linked Immuno Sorbent Assay), and LOCI (Luminescence Oxygen Channeling Immunoasssay). General protocols for suitable RIA and ELISA assays are found in, e.g., WO09/030738 on p. 116-118.

The conjugation of the GLP-1 analogue and the activated side chain is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate).

Herein, the naming of the peptides or proteins is done according to the following principles: The names are given as mutations and modifications (such as acylations) relative to the parent peptide or protein such as human GLP-1 (GLP-1(7-37)). For the naming of the acyl moiety, the naming is done according to IUPAC nomenclature and in other cases as peptide nomenclature. For example, naming the acyl moiety:

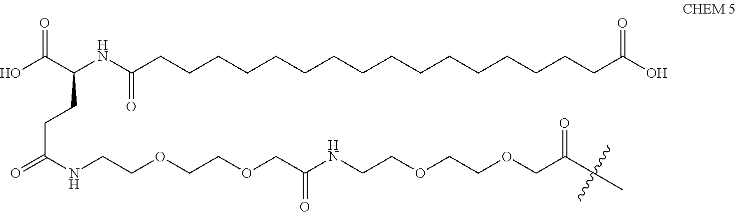 [SMILES: *C(=O)COCCOCCNC(=O)COCCOCCNC(=O)CC[C@H](NC(=O)CCCCCCCCCCCCCCCCC(=O)O)C(=O)O]

may e.g. be “(17-Carboxyheptadecanoylamino)-4(S)-carboxybutyrylamino]ethoxy)-ethoxy]acetylamino)ethoxy]ethoxy)acetyl”, “octadecanedioyl-γGlu-OEG-OEG”, “octadecanedioyl-gGlu-OEG-OEG”, “octadecanedioyl-gGlu-2xOEG”, or “17-carboxyheptadecanoyl-γGlu-OEG-OEG”, wherein OEG is short hand notation for the amino acid residue, 8-amino-3,6-dioxaoctanoic acid, —NH(CH₂)₂O(CH₂)₂OCH₂CO—, and γGlu (or gGlu) is short hand notation for the amino acid gamma L-glutamic acid moiety.

One example is the acylated GLP-1 peptide of example 4 (see also example 4 in patent application WO 2006/097537) with the sequence/structure given below, which is named “N{Epsilon-26}-[2-[2-[2-[[2-[2-[2-[[(4S)-4-carboxy-4-(17-carboxyheptadecanoylamino)-butanoyl]amino]ethoxy]ethoxy]acetyl]amino]ethoxy]ethoxy]acetyl]-[Aib8,Arg34]-GLP-1-(7-37)-peptide” to indicate that the amino acid alanine (abbreviated Ala or A) in position 8 in GLP-1(7-37) has been mutated to the non-proteinogenic amino acid α-aminoisobutyric acid (abbreviated Aib), the amino acid in position 34 in GLP-1(7-37) has been mutated to arginine (abbreviated Arg or R) and the amino acid in position 26, lysine (abbreviated Lys or K) as in GLP-1(7-37), has been modified by acylation on the epsilon nitrogen in the lysine residue of position 26, denoted N{Epsilon-26}, by the residue 2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]-acetyl (alternatively denoted e.g. “(17-Carboxyheptadecanoylamino)-4(S)-carboxybutyrylamino]ethoxy)ethoxy]acetylamino)ethoxy]ethoxy)acetyl” or “octadecanedioyl-γGlu-OEG-OEG”). Alternatively the peptide may e.g. be named ““N-ε²⁶-[2-(2-[2-(2-[2-(2-[4-(17-Carboxy-heptadecanoylamino)-4(S)-carboxybutyrylamino]ethoxy)ethoxy]acetylamino)ethoxy]ethoxy)-acetyl][Aib8,Arg34]GLP-1(7-37) peptide””. Asterisks in the formula below indicate that the residue in question is different (i.e. mutated) as compared to GLP-1(7-37). In addition, the acylated GLP-1 peptides of the pharmaceutical composition of the invention may also be named according to IUPAC nomenclature (OpenEye, IUPAC style) as e.g. “N^{e26}[(S)-(22,40-dicarboxy-10,19,24-trioxo-3,6,12,15-tetraoxa-9,18,23-triazatetracontan-1-oyl)][Aib⁸, Arg³⁴]GLP-1-(7-37) peptide”. Herein, the term “amino acid residue” is an amino acid from which a hydroxy group has been removed from a carboxy group and/or from which a hydrogen atom has been removed from an amino group.

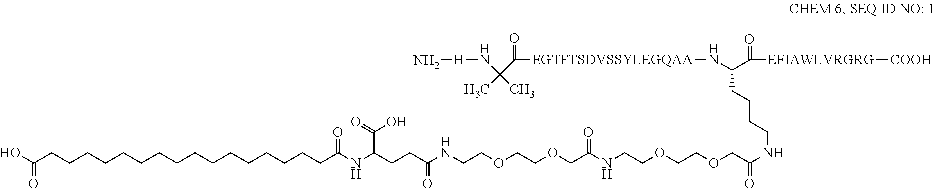

In the sequence listing, the first amino acid residue of SEQ ID NO: 1 (histidine) is assigned no. 1. However,—according to established practice in the art—this histidine residue is herein referred to as no. 7, and subsequent amino acid residues are numbered accordingly, ending with glycine no. 37. Therefore, generally, any reference herein to an amino acid residue number or a position number of the GLP-1(7-37) sequence is to the sequence starting with His at position 7 and ending with Gly at position 37.

In one aspect of the invention, the active peptide ingredient is an insulin peptide.

The term “insulin peptide” as used herein means a peptide which is either human insulin or an analog or a derivative thereof with insulin activity.

The term “human insulin” as used herein means the human insulin hormone whose structure and properties are well-known. Human insulin has two polypeptide chains, named the A-chain and the B-chain. The A-chain is a 21 amino acid peptide and the B-chain is a 30 amino acid peptide, the two chains being connected by disulphide bridges: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain.

In the human body, the hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains.

An insulin peptide for use in the invention has at least 0.01% Insulin Receptor affinity as defined below. It is thus known that even at very low insulin receptor affinity of e.g. a human insulin peptide, in vivo biological activity may be attained for the same human insulin analogue (as e.g. illustrated in Diabetes 39, 1033-1039 (1990), Ribel et al.).

The term “insulin analogue” as used herein means a modified human insulin wherein one or more amino acid residues of the insulin have been substituted by other amino acid residues and/or wherein one or more amino acid residues have been deleted from the insulin and/or wherein one or more amino acid residues have been added and/or inserted to the insulin.

In one aspect an insulin analogue comprises 10 amino acid modifications (substitutions, deletions, additions (including insertions) and any combination thereof) or less relative to human insulin, alternatively 9, 8, 7, 6, 5, 4, 3 or 2 modifications or less, yet alternatively 1 modification relative to human insulin.

Modifications in the insulin molecule are denoted stating the chain (A or B), the position, and the one or three letter code for the amino acid residue substituting the native amino acid residue.

By “connecting peptide” or “C-peptide” is meant a connection moiety “C” of the B-C-A polypeptide sequence of a single chain proinsulin-molecule. In the human insulin chain, the C-peptide connects position 30 of the B chain and position 1 of the A chain and is 35 amino acid residue long. The connecting peptide includes two terminal dibasic amino acid sequence, e.g., Arg-Arg and Lys-Arg which serve as cleavage sites for cleavage off of the connecting peptide from the A and B chains to form the two-chain insulin molecule.

By “desB30” or “B(1-29)” is meant a natural insulin B chain or an analogue thereof lacking the B30 amino acid and “A(1-21)” means the natural insulin A chain. Thus, e.g., A14Glu,B25His,desB30 human insulin is an analogue of human insulin where the amino acid in position 14 in the A chain is substituted with glutamic acid, the amino acid in position 25 in the B chain is substituted with histidine, and the amino acid in position 30 in the B chain is deleted.

Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, terms like A21A, A21G and A21Q designates that the amino acid in the A21 position is A, G and Q, respectively. Using the three letter codes for amino acids, the corresponding expressions are A21Ala, A21Gly and A21 Gln, respectively.

Examples of insulin analogues are such wherein the amino acid in position A14 is Glu, the amino acid in position B25 is His and which optionally further comprises one or more additional mutations. Further examples of insulin analogues are the deletion analogues, e.g., analogues where the B30 amino acid in human insulin has been deleted (des(B30) human insulin). Insulin analogues wherein the A-chain and/or the B-chain have an N-terminal extension and insulin analogues wherein the A-chain and/or the B-chain have a C-terminal extension such as with two arginine residues added to the C-terminal of the B-chain are also examples of insulin analogues.

The term “insulin derivative” as used herein means a chemically modified parent insulin or an analogue thereof, wherein the modification(s) are in the form of attachment of amides, carbohydrates, alkyl groups, acyl groups, esters, PEGylations, and the like.

In one aspect of the invention, the modification(s) include attachment of a side chain to human insulin or an analogue thereof. In a particular aspect, the side chain is capable of forming non-covalent aggregates with albumin, thereby promoting the circulation of the derivative with the blood stream, and also having the effect of protracting the time of action of the derivative, due to the fact that the aggregate of the insulin-derivative and albumin is only slowly disintegrated to release the active peptide ingredient. Thus, the substituent, or side chain, as a whole is preferably referred to as an albumin binding moiety. In particular aspects, the side chain has at least 10 carbon atoms, or at least 12, 14, 16, 18, 20, 22, or at least 24 carbon atoms. In further particular aspects, the side chain may further include at least 5 hetero atoms, in particular O and N, for example at least 7, 9, 10, 12, 15, 17, or at least 20 hetero atoms, such as at least 1, 2, or 3 N-atoms, and/or at least 3, 6, 9, 12, or 15 O-atoms.

In another particular aspect the albumin binding moiety comprises a portion which is particularly relevant for the albumin binding and thereby the protraction, which portion may accordingly be referred to as a “protracting moiety”. The protracting moiety may be at, or near, the opposite end of the albumin binding moiety, relative to its point of attachment to the peptide.

In a still further particular aspect the albumin binding moiety comprises a portion in between the protracting moiety and the point of attachment to the peptide, which portion may be referred to as a “linker”, “linker moiety”, “spacer”, or the like. The linker may be optional, and hence in that case the albumin binding moiety may be identical to the protracting moiety.

In particular aspects, the albumin binding moiety and/or the protracting moiety is lipophilic, and/or negatively charged at physiological pH (7.4).

The albumin binding moiety, the protracting moiety, or the linker may e.g. be covalently attached to a lysine residue of human insulin or an insulin analogue by acylation.

In one aspect, an active ester of the albumin binding moiety, preferably comprising a protracting moiety and a linker, is covalently linked to an amino group of a lysine residue, preferably the epsilon amino group thereof, under formation of an amide bond (this process being referred to as acylation).

Unless otherwise stated, when reference is made to an acylation of a lysine residue, it is understood to be to the epsilon-amino group thereof.

For the present purposes, the terms “albumin binding moiety”, “protracting moiety”, and “linker” may include the unreacted as well as the reacted forms of these molecules. Whether or not one or the other form is meant is clear from the context in which the term is used.

For the attachment to human insulin or the insulin analogue, the acid group of the fatty acid, or one of the acid groups of the fatty diacid, forms an amide bond with the epsilon amino group of a lysine residue in human insulin or the insulin analogue, preferably via a linker.

The term “fatty diacid” refers to fatty acids as defined above but with an additional carboxylic acid group in the omega position. Thus, fatty diacids are dicarboxylic acids.

Each of the two linkers of the derivative of the invention may comprise the following first linker element:

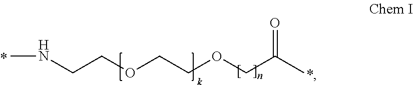

wherein k is an integer in the range of 1-5, and n is an integer in the range of 1-5.

In a particular aspect, when k=1 and n=1, this linker element may be designated OEG, or a di-radical of 8-amino-3,6-dioxaoctanic acid, and/or it may be represented by the following formula:

NH—(CH₂)₂—O—(CH₂)₂—O—CH₂—CO—*. Chem II:

In another particular aspect, each linker of the derivative of the invention may further comprise, independently, a second linker element, preferably a Glu di-radical, such as Chem III and/or Chem IV:

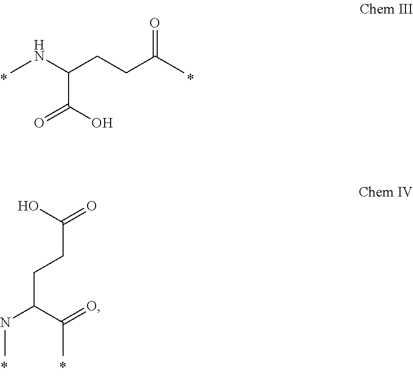

wherein the Glu di-radical may be included p times, where p is an integer in the range of 1-3.

Chem III may also be referred to as gamma-Glu, or briefly γGlu, due to the fact that it is the gamma carboxy group of the amino acid glutamic acid which is here used for connection to another linker element, or to the epsilon-amino group of lysine. As explained above, the other linker element may, for example, be another Glu residue, or an OEG molecule. The amino group of Glu in turn forms an amide bond with the carboxy group of the protracting moiety, or with the carboxy group of, e.g., an OEG molecule, if present, or with the gamma-carboxy group of, e.g., another Glu, if present.

Chem IV may also be referred to as alpha-Glu, or briefly aGlu, or simply Glu, due to the fact that it is the alpha carboxy group of the amino acid glutamic acid which is here used for connection to another linker element, or to the epsilon-amino group of lysine.

The above structures of Chem III and Chem IV cover the L-form, as well as the D-form of Glu. In particular aspects, Chem III and/or Chem IV is/are, independently, a) in the L-form, or b) in the D-form.

In still further particular aspects the linker has a) from 5 to 41 C-atoms; and/or b) from 4 to 28 hetero atoms.

Herein, the naming of the insulin peptides is done as explained earlier in the application in connection with GLP-1 peptides: The names are given as mutations and modifications (such as acylations) relative to the parent peptide, i.e. human insulin.

One example is the acylated insulin peptide insulin 1 of example 3 (see also example 9 in patent application WO 2009/115469) with the sequence/structure given below, which is named “N{Epsilon-B29}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[GluA14,HisB25],des-ThrB30-Insulin(human)” to indicate that the amino acid tyrosine in position 14 in the A-chain of human insulin has been mutated to the amino acid glutamic acid (abbreviated Glu or E), the amino acid phenylalanine in position 25 in the B-chain of human insulin has been mutated to histidine (abbreviated His or H), the amino acid threonine in position 30 in the B-chain of human insulin has been deleted and the amino acid in position 29 of the B-chain of human insulin, lysine (abbreviated Lys or K), has been modified by acylation on the epsilon nitrogen in the lysine residue, denoted N{Epsilon-B29}, by the residue [2-[2-[2-[[2-[2-[2-[[(4S)-4-carboxy-4-(17-carboxyheptadecanoylamino)butanoyl]-amino]ethoxy]ethoxy]acetyl]amino]ethoxy]ethoxy]acetyl] (alternatively denoted “octadecanedioyl-γGlu-OEG-OEG”).

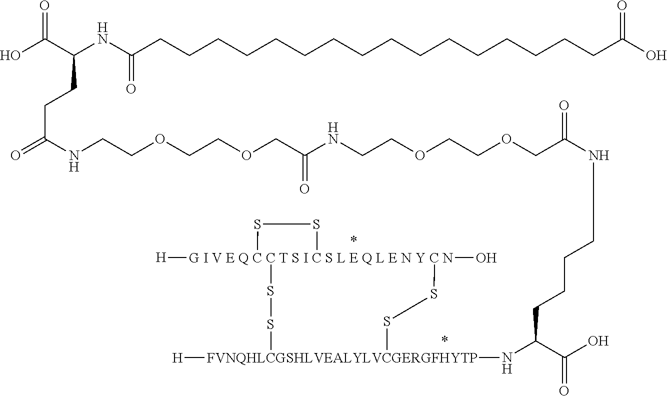

CHEM 7, SEQ ID NO: 2 and 3 In addition, the acylated insulin of the pharmaceutical composition of the invention may also be named “A14E, B25H, B29K(N^{ε}Octadecanedioyl-γGlu-OEG-OEG), desB30 human insulin” or according to IUPAC nomenclature (Open Eye, IUPAC style) as e.g. “N{Epsilon-B29}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[GluA14,HisB25],des-ThrB30-Insulin(human)”.

A non-limiting example of a derivative of an insulin analogue for use in solid oral pharmaceutical compositions according to the invention includes N{Epsilon-B29}-[2-[2-[2-[[2-[2-[2-[[(4S)-4-carboxy-4-(17-carboxyheptadecanoylamino)butanoyl]amino]ethoxy]ethoxy]-acetyl]amino]ethoxy]ethoxy]acetyl][GluA14,HisB25],des-ThrB30-Insulin(human).

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate).

As used herein, a “therapeutically effective amount of an enhancer” refers to an amount of enhancer that allows for uptake of therapeutically effective amounts of the therapeutically active peptide ingredient via oral administration. It has been shown that the effectiveness of an enhancer in improving the gastrointestinal absorption of poorly absorbed drugs is dependent on the site of administration, the site of optimum delivery being dependent on the drug and enhancer.

### EMBODIMENTS OF THE INVENTION

The following are non-limiting examples of embodiments of the invention:

1. Solid oral pharmaceutical composition comprising
a) an active peptide ingredient which is an insulin peptide or GLP-1 peptide,
b) a salt of capric acid such as sodium caprate,

### c) Bowman-Birk Inhibitor (BBI), and

d) a BBI solubilizing agent, such as a sugar alcohol, such as sorbitol.
2. The solid oral pharmaceutical composition of embodiment 1, wherein at least 10 mg salt of capric acid and at least 1 mg BBI is present per dosage form.
3. The solid oral pharmaceutical composition of any one of the preceding embodiments, comprising 10 mg-450 mg salt of capric acid and 1 mg-200 mg BBI.
4. The solid oral pharmaceutical composition of any one of the preceding embodiments, comprising 10 mg-450 mg salt of capric acid and 10 mg-200 mg BBI.
5. The solid oral pharmaceutical composition of any one of embodiments 1-3, comprising 10 mg-275 mg salt of capric acid and 1 mg-200 mg BBI.
6. The solid oral pharmaceutical composition of any one of the preceding embodiments, comprising 10 mg-275 mg salt of capric acid and 10 mg-200 mg BBI.
7. The solid oral pharmaceutical composition of any one of embodiments 1-4, comprising 10 mg-450 mg salt of capric acid and 10 mg-50 mg BBI.
8. The solid oral pharmaceutical composition of any one of the preceding embodiments, comprising 10 mg-275 mg salt of capric acid and about 50 mg BBI.
9. The solid oral pharmaceutical composition of any one of the preceding embodiments, comprising about 275 mg salt of capric acid and from 10 mg-50 mg BBI.
10. The solid oral pharmaceutical composition of any one of the preceding embodiments, comprising 10 mg-400 mg BBI solubilizing agent.
11. The solid oral pharmaceutical composition of any one of the preceding embodiments, comprising 100 mg-275 mg sorbitol.
12. The solid oral pharmaceutical composition of any one of the preceding embodiments, wherein the solid oral pharmaceutical composition is in the form of a powder or granulate comprised in a capsule.
13. The solid oral pharmaceutical composition of embodiment 12, wherein the capsule comprises up to 1000 mg powder, such as up to 650 mg powder.
14. The solid oral pharmaceutical composition of any one of embodiments 1-13 comprising:

| | |
|---|---|
| 1.4% (w/w) | Insulin peptide |
| 8.3% (w/w) | BBI |
| 45.8% (w/w) | Sodium decanoate (Sodium caprate) |
| 43.9% (w/w) | Sorbitol |
| 0.5% (w/w) | Magnesium stearate |

15. The solid oral pharmaceutical composition of any one of embodiments 1-13 comprising:

| | |
|---|---|
| 1.4% (w/w) | Insulin peptide |
| 1.7% (w/w) | BBI |
| 45.8% (w/w) | Sodium caprate |
| 50.6% (w/w) | Sorbitol |
| 0.5% (w/w) | Magnesium stearate |

16. The solid oral pharmaceutical composition of any one of embodiments 1-13 comprising:

| | |
|---|---|
| 1.4% (w/w) | Insulin peptide |
| 4.2% (w/w) | BBI |
| 45.8% (w/w) | Sodium caprate |
| 48.1% (w/w) | Sorbitol |
| 0.5% (w/w) | Magnesium stearate |

17. The solid oral pharmaceutical composition of any one of embodiments 1-13 comprising:

| | |
|---|---|
| 1.4% (w/w) | Insulin peptide |
| 33.3% (w/w) | BBI |
| 45.8% (w/w) | Sodium caprate |
| 18.9% (w/w) | Sorbitol |
| 0.5% (w/w) | Magnesium stearate |

18. The solid oral pharmaceutical composition of any one of embodiments 1-13 comprising:

| | |
|---|---|
| 1.4% (w/w) | Insulin peptide |
| 8.3% (w/w) | BBI |
| 25.0% (w/w) | Sodium caprate |
| 64.8% (w/w) | Sorbitol |
| 0.5% (w/w) | Magnesium stearate |

19. The solid oral pharmaceutical composition of any one of embodiments 1-13 comprising:

| | |
|---|---|
| 1.4% (w/w) | Insulin peptide |
| 8.3% (w/w) | BBI |
| 58.3% (w/w) | Sodium caprate |
| 31.4% (w/w) | Sorbitol |
| 0.5% (w/w) | Magnesium stearate |

20. The solid oral pharmaceutical composition of any one of embodiments 1-13 comprising:

| | |
|---|---|
| 1.4% (w/w) | Insulin peptide |
| 8.3% (w/w) | BBI |
| 75.0% (w/w) | Sodium caprate |
| 14.8% (w/w) | Sorbitol |
| 0.5% (w/w) | Magnesium stearate |

21. The solid oral pharmaceutical composition of any one of embodiments 1-13 comprising:

| | |
|---|---|
| 1.4% (w/w) | Insulin peptide |
| 1.7% (w/w) | BBI |
| 75.0% (w/w) | Sodium caprate |
| 21.4% (w/w) | Sorbitol |
| 0.5% (w/w) | Magnesium stearate |

22. The solid oral pharmaceutical composition of any one of embodiments 1-13 comprising:

| | |
|---|---|
| 3.4% (w/w) | GLP-1 peptide |
| 8.3% (w/w) | BBI |
| 45.8% (w/w) | Sodium decanoate (Sodium caprate) |
| 41.9% (w/w) | Sorbitol |
| 0.5% (w/w) | Magnesium stearate |

23. The solid oral pharmaceutical composition of any one of embodiments 1-13 comprising:

| | |
|---|---|
| 3.3% (w/w) | GLP-1 peptide |
| 1.7% (w/w) | BBI |
| 45.8% (w/w) | Sodium caprate |
| 48.6% (w/w) | Sorbitol |
| 0.5% (w/w) | Magnesium stearate |

24. The solid oral pharmaceutical composition of any one of embodiments 1-13 comprising:

| | |
|---|---|
| 3.3% (w/w) | GLP-1 peptide |
| 33.3% (w/w) | BBI |
| 45.8% (w/w) | Sodium caprate |
| 17.0% (w/w) | Sorbitol |
| 0.5% (w/w) | Magnesium stearate |

25. The solid oral pharmaceutical composition of any one of embodiments 1-13 comprising:

| | |
|---|---|
| 150 mg | sodium caprate |
| 50 mg | BBI |
| 389 mg | sorbitol |

26. The solid oral pharmaceutical composition of any one of embodiments 1-13 comprising:

| | |
|---|---|
| 275 mg | sodium caprate |
| 10 mg | BBI |
| 315 mg | sorbitol |

27. The solid oral pharmaceutical composition of any one of embodiments 1-13 comprising:

| | |
|---|---|
| 275 mg | sodium caprate |
| 25 mg | BBI |
| 300 mg | sorbitol |

28. The solid oral pharmaceutical composition of any one of embodiments 1-13 comprising:

| | |
|---|---|
| 275 mg | sodium caprate |
| 50 mg | BBI |
| 275 mg | sorbitol |

29. The solid oral pharmaceutical composition of any one of embodiments 1-13 comprising:

| | |
|---|---|
| 275 mg | sodium caprate |
| 200 mg | BBI |
| 125 mg | sorbitol |

30. The solid oral pharmaceutical composition of any one of embodiments 1-13 comprising:

| | |
|---|---|
| 275 mg | sodium caprate |
| 50 mg | BBI |
| 275 mg | microcrystaline cellulose |

31. The solid oral pharmaceutical composition of any one of embodiments 1-13 comprising:

| | |
|---|---|
| 350 mg | sodium caprate |
| 50 mg | BBI |
| 200 mg | sorbitol |

32. The solid oral pharmaceutical composition of any one of embodiments 1-13 comprising:

| | |
|---|---|
| 450 mg | sodium caprate |
| 10 mg | BBI |
| 140 mg | sorbitol |

33. The solid oral pharmaceutical composition of any one of embodiments 1-13 comprising:

| | |
|---|---|
| 450 mg | sodium caprate |
| 50 mg | BBI |
| 100 mg | sorbitol |

34. The solid oral pharmaceutical composition of embodiment 12 or 13, wherein the capsule is a hard capsule.
35. The solid oral pharmaceutical composition of any one of the preceding embodiments, further comprising a coating
36. The solid oral pharmaceutical composition of embodiment 15, wherein the coating is an enteric coating.
37. The solid oral pharmaceutical composition of any one of the preceding embodiments, wherein BBI is isolated from plants.
38. The solid oral pharmaceutical composition of any one of embodiments 1-17, wherein BBI is isolated from legumes
39. The solid oral pharmaceutical composition of any one of embodiments 1-17, wherein BBI is isolated from soy beans
40. The solid oral pharmaceutical composition of any one of the preceding embodiments, wherein BBI is freeze- or spray dried
41. The solid oral pharmaceutical composition of any one of the preceding embodiments, wherein the purity of BBI is at least 90% pure.
42. The solid oral pharmaceutical composition of any one of embodiments 1-16 or 20-21, wherein BBI is recombinantly produced.
43. The solid oral pharmaceutical composition of any one of the preceding embodiments, wherein the insulin peptide or the GLP-1 peptide is acylated.
44. The solid oral pharmaceutical composition of any one of the preceding embodiments, wherein the active peptide ingredient is an insulin peptide which is acylated.
45. The solid oral pharmaceutical composition of any one of the preceding embodiments, wherein the insulin peptide is N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]-acetyl]-[GluA14,HisB25],des-ThrB30-Insulin(human).
46. The solid oral pharmaceutical composition of any one of embodiments 1-44, wherein the insulin peptide is selected from the group consisting of: N{Epsilon-B29}-[2-[2-[2-[[2-[2-[2-[[(4S)-4-carboxy-4-(17-carboxyheptadecanoylamino)-butanoyl]amino]ethoxy]ethoxy]acetyl]amino]ethoxy]ethoxy]acetyl]-[GluA14,HisB25],des-ThrB30-Insulin(human); N{A1}, N{A1}-dimethyl, N{B1}, N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl][GluA14,HisB25],des-ThrB30-Insulin(human); N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-[GluA14,HisB16,HisB25],des-ThrB30-Insulin(human); N{Epsilon-B29}-[(4S)-4-carboxy-4-(17-carboxyheptadecanoylamino)butanoyl]-[GluA14,HisB25],des-ThrB27,ThrB30-Insulin(human); N{Epsilon-B29}-tetradecanoyl-des-ThrB30-Insulin(human); N{Epsilon-B29}-[(4S)-4-carboxy-4-(15-carboxypentadecanoylamino)butanoyl]-des-ThrB30-Insulin(human), N{Epsilon-B29}-[(4S)-4-carboxy-4-(15-carboxypentadecanoylamino)butanoyl]-[GluA14, HisB16,HisB25],des-ThrB30-Insulin(human); N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[CysA10,GluA14,CysB4,HisB25],des-ThrB30-Insulin(human); and N{Epsilon-B29}-[(4S)-4-carboxy-4-(19-carboxynonadecanoylamino)butanoyl]-[CysA10, GluA14,CysB3, HisB25],des-ThrB27,ThrB30-Insulin(human).
47. The solid oral pharmaceutical composition of any one of embodiments 1-44, wherein the active peptide ingredient is a GLP-1 peptide which is acylated.
48. The solid oral pharmaceutical composition of embodiment 47, wherein the GLP-1 peptide is selected from N{Epsilon-26}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[Aib8,Arg34]-GLP-1-(7-37)-peptide; N{Epsilon-18}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]-acetyl], N{Epsilon-26}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib8,Lys18,Glu22,Gln34]-GLP-1-(7-37)-peptide; N{Epsilon-26}-[(2S)-2-amino-6-[[(2S)-2-amino-6-[[(4S)-4-carboxy-4-(15-carboxypentadecanoylamino)butanoyl]amino]hexanoyl]-amino]hexanoyl], N{Epsilon-37}-[(2S)-2-amino-6-[[(2S)-2-amino-6-[[(4S)-4-carboxy-4-(15-carboxypentadecanoylamino)butanoyl]amino]hexanoyl]amino]hexanoyl]-[Aib8,Arg34,Lys37]-GLP-1-(7-37)-peptide; N{Epsilon-18}-[2-[2-[2-[[2-[2-[2-[[(4S)-4-carboxy-4-[10-(4-carboxy- phenoxy)decanoylamino]butanoyl]amino]ethoxy]ethoxy]acetyl]amino]ethoxy]ethoxy]acetyl], —N{Epsilon-26}-[2-[2-[2-[[2-[2-[2-[[(4S)-4-carboxy-4-[10-(4-carboxyphenoxy)decanoylamino]-butanoyl]amino]ethoxy]ethoxy]acetyl]amino]ethoxy]ethoxy]acetyl]-[Aib8,Lys18,Glu22,Arg34]-GLP-1-(7-37)-peptide; N{Epsilon-18}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]-acetyl], N{Epsilon-26}-[2-[2-[2-[[2-[2-[2-[[(4S)-4-carboxy-4-(13-carboxytridecanoylamino)-butanoyl]amino]ethoxy]ethoxy]acetyl]amino]ethoxy]ethoxy]acetyl]-[Aib8,Lys18,Glu22,Arg34]-GLP-1-(7-37)-peptide; N{Epsilon-18}-[2-[2-[2-[[2-[2-[2-[[(4S)-4-carboxy-4-[10-(4-carboxy- phenoxy)decanoylamino]butanoyl]amino]ethoxy]ethoxy]acetyl]amino]ethoxy]ethoxy]-acetyl], N{Epsilon-26}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib8,Lys18,Gln34]-GLP-1-(7-37)-peptide; N{Epsilon-26}-[2-[2-[2-[[2-[2-[2-[[(4S)-4-carboxy-4-(15-carboxy- pentadecanoylamino)butanoyl]amino]ethoxy]ethoxy]acetyl]amino]ethoxy]ethoxy]acetyl]-[Aib8,Arg34]-GLP-1-(7-37)-peptide; N{Epsilon-26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]-acetyl]-[Aib8,His31,Gln34]-GLP-1-(7-37)-peptide; N{Epsilon-26}-[2-[2-[2-[[2-[2-[2-[[(4S)-4-carboxy-4-[10-(4-carboxyphenoxy)decanoylamino]butanoyl]amino]ethoxy]ethoxy]acetyl]-amino]ethoxy]ethoxy]acetyl], N{Epsilon-37}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]-acetyl]-[Aib8,Arg34,Lys37]-GLP-1-(7-37)-peptide; and N{Epsilon-26}-[2-[2-[2-[[(4S)-4-carboxy-4-[[2-[2-[2-(13-carboxytridecanoylamino)ethoxy]ethoxy]acetyl]amino]butanoyl]-amino]ethoxy]ethoxy]acetyl], N{Epsilon-37}-[2-[2-[2-[[(4S)-4-carboxy-4-[[2-[2-[2-(13-carboxytridecanoylamino)ethoxy]ethoxy]acetyl]amino]butanoyl]amino]ethoxy]ethoxy]acetyl]-[Aib8,Arg34,Lys37]-GLP-1-(7-37)-peptide.
49. The solid oral pharmaceutical composition of embodiment 48, wherein the GLP-1 peptide is N{Epsilon-26}-[2-[2-[2-[[2-[2-[2-[[(4S)-4-carboxy-4-(17-carboxyheptadecanoylamino)-butanoyl]amino]ethoxy]ethoxy]acetyl]amino]ethoxy]ethoxy]acetyl]-[Aib8,Arg34]-GLP-1-(7-37)-peptide.
50. The solid oral pharmaceutical composition of any one of the preceding embodiments in which the ratio (w/w) of the salt of capric acid to BBI is 45:1 or less.
51. The solid oral pharmaceutical composition of any one of the preceding embodiments in which the ratio (w/w) of the salt of capric acid to BBI is 30:1 or less.
52. The solid oral pharmaceutical composition of any one of the preceding embodiments in which the ratio (w/w) of the salt of capric acid to BBI is 9:1 or less.
53. The solid oral pharmaceutical composition of any one of the preceding embodiments in which the ratio (w/w) of the salt of capric acid to BBI is 5.5:1 or less.
54. The solid oral pharmaceutical composition of any one of the preceding embodiments in which the ratio (w/w) of the salt of capric acid to BBI is about 1:1.
55. The solid oral pharmaceutical composition of any one of the preceding embodiments, wherein the solid oral pharmaceutical composition is a powder contained in a tablet.
56. The solid oral pharmaceutical composition of any one of the preceding embodiments, wherein the solid oral pharmaceutical composition is in the form of a powder comprised in a capsule.
57. The solid oral pharmaceutical composition of any one of the preceding embodiments, wherein the solid oral pharmaceutical composition is in the form of a granulate comprised in a capsule.

### EXAMPLES

### General Procedures

The following examples are offered by way of illustration, not by limitation.

### LIST OF ABBREVIATIONS

HCl: hydrochloric acid,

MeCN: acetonitrile,

OEG: [2-(2-aminoethoxy)ethoxy]ethylcarbonyl,

RPC: reverse phase chromatography,

RT: room temperature,

TFA: trifluoroacetic acid,

DMSO: Dimethyl sulfoxide

GI: gastro intestinal,

TRIS: tris(hydroxymethyl)aminomethane,

CH3CN: Acetonitril,

BSA: bovine serum albumin

HEPES: 4-(2-hydroxyethyl)-1-piperazineethanesulfonic acid

HBSS: Hank's balanced salt solution

Tween 20: Polyethylene glycol sorbitan monolaurate20

LOCI assay: Luminescence Oxygen Channeling Immunoasssay

HPLC: High-performance liquid chromatography,

FPLC: Fast protein liquid chromatography,

RP: Reverse phase,

UV: Ultraviolet (light),

LC-MS: Liquid chromatography-mass spectrometry,

MALDI MS: Matrix assisted laser desorption mass spectrometry

NMR: Nuclear magnetic resonance,

TLC: thin layer chromatography,

GLP-1: Glucagon-like peptide-1,

GI juice: gastro-intestinal juice,

HI: Human insulin,

BBI: Bowman-Birk inhibitor

### General Methods of Preparation

Insulin peptides and GLP-1 peptides were prepared according to methods known to the person skilled in the art as e.g. described in the examples of WO 2009/115469, WO.2006/097537 and WO 2011/080103.

In general, insulin peptides and GLP-1 peptides may be prepared by recombinant expression, for example in E. coli (FEBS Lett. 1997, 402, 124) or S. cerevisae (Protein Sci. 2013, 22, 296-305). Alternatively insulin peptides and GLP-1 peptides may be prepared by total chemical synthesis (J Am Chem Soc. 2013, 135, 3173-85.) using either solid phase or liquid phase synthesis; or a combination of recombinant expression and chemical synthesis (as e.g. described in WO 2009/083549). Modification by insulin and GLP-1 peptides was performed by standard acylation technology (as e.g. described in WO 2010/029159 and WO 2013/098191).

Capric acid was manufactured using palm oil as the starting material. The capric acid was mixed with a NaOH solution. This solution was spray dried to obtain a sodium caprate powder. Finally, the spray dried sodium caprate was dry granulated using a roller compactor.

Bowman-Birk inhibitor was obtained from Sigma-Aldrich and further purified before use (see example 1).

In general, Bowman-Birk inhibitor may be purified from plant material, or may be prepared using recombinant expression systems as described in the patent description.

### Purification

Typical purification procedures for insulin peptides or GLP-1 peptides:

The HPLC system used was a Gilson system consisting of the following: Model 215 Liquid handler, Model 322-H2 Pump and a Model 155 UV Dector. Detection was typically at 210 nm and 280 nm.

The Âkta Purifier FPLC system (GE) consisted of the following: Model P-900 Pump, Model UV-900 UV detector, Model pH/C-900 pH and conductivity detector, Model Frac-950 Fraction collector. UV detection was typically at 214 nm, 254 nm and 276 nm.

Acidic HPLC:

Column: Macherey-Nagel SP 250/21 Nucleusil 300-7 C4

Flow: 8 ml/min

Buffer A: 0.1% TFA in acetonitrile

Buffer B: 0.1% TFA in water.

Gradient: 0.0-5.0 min: 10% A

5.00-30.0 min: 10% A to 90% A

30.0-35.0 min: 90% A

35.0-40.0 min: 100% A

Neutral HPLC:

Column: Phenomenex, Jupiter, C4 5 μm 250×10.00 mm, 300 Å

Flow: 6 ml/min

Buffer A: 5 mM TRIS, 7.5 mM (NH₄)₂SO₄, pH=7.3, 20% CH₃CN

Buffer B: 60% CH₃CN, 40% water

Gradient: 0-5 min: 10% B

5-65 min: 10-90% B

65-69 min: 90% B

69-80 min: 90% B

Desalting:

Column: HiPrep 26/10

Flow: 10 ml/min, 6 column volumes

Buffer: 10 mM NH₄HCO₃

### General Methods of Detection and Characterisation of Insulin/GLP-1 Peptides and BBI

The identity and purity of the insulin and/or GLP-1 peptides and/or BBI was typically determined by RP-HPLC analysis; LC-MS analysis and/or MALDI MS analysis. RP-HPLC system consisted of Acquity UPLC components: autosampler (Model Acq-SM), pump (Model Acq-BSM), column oven (Model Acq-SM) and detector (Model Acq-TUV; Waters, Milford, Mass.). Variety of RP-HPLC columns and buffer compositions may be used in this system to achieve appropriate separation; for example, Acquity BEH 1.7 μM C18 1×50 mm column (Waters) was applied using a linear gradient of acetonitrile in 0.2 M sodium sulfate, 0.04M sodium phosphate, pH=7.2. Peaks were typically detected by UV absorption for example at 220 nm and quantified using an appropriate standard (such as human insulin for insulin analysis, etc).

LC-MS system consisted of Waters Acquity UPLC system (Waters) consisting of an autosampler (Model Acq-SM), pump (Model Acq-BSM), column oven (Model Acq-SM), detector (Model Acq-TUV) and LTQ Orbitrap XL (Thermo Fisher). RP-HPLC separation was achieved using a linear gradient of acetonitrile in 0.1% formic acid using CSH C18 column (Waters, 1×150 mm) with a flow rate of 0.1 ml/min at 45° C.

Insulin peptides, GLP-1 peptides and BBI were typically described as acids, however, it is understood that different salt forms of these compounds were present based on the purification procedure used and/or buffer applied when making stock solutions. Such salts include but are not limited to sodium salt, potassium salt, ammonium salt, formate salt, acetate salt, trifluoroacetate salt, phosphate salt, bicarbonate salt etcetera.

### Example 1

### Purification of BBI

Purity (measured using the method described in example 2) of BBI (all BBI related species) obtained from Sigma ranged from 50-70% depending on the batch. BBI was therefore further purified before use according to the following RP-HPLC procedure:

Equipment: Akta

Column: Gemini_NX_5β_C18_110 Å column (Phenomenex, Torrance, Calif., USA, 176.7 ml)

Equilibration solvent: 5 v/v % acetonitrile, 0.1 v/v % TFA

Elution solvent: 0.1 v/v % TFA in acetonitrile

Flow: 10 ml/min

Gradient: 0%-20% elution solvent in 25 column volumes

Fractions containing pure BBI were combined and diluted 2-fold with water prior to lyophilization. Typically, all impurities not related to BBI were removed by this purification procedure leaving essentially pure BBI containing several truncated forms as determined by LC-MS analysis. BBI purity (all BBI related species) after purification was at least 90%.

### Example 2

### Analysis of BBI—Purity, Integrity

Fractions were analysed using UPLC system (Waters, Milford, Mass.) equipped with BEH Shield RP18 2.1 mm×150 mm-1.7 μm, 130 Å column (Waters) and eluted with a linear gradient of acetonitrile in 0.1% TFA at 60° C. with following gradient:

| Time (min) | Flow (mL/min) | % A | % B |
|---|---|---|---|
| 0.00 | 0.400 | 95.0 | 5.0 |
| 2.00 | 0.400 | 95.0 | 5.0 |
| 20.00 | 0.400 | 60.0 | 40.0 |
| 25.00 | 0.400 | 10.0 | 90.0 |

BBI eluted as a single peak in this method. The purity of BBI was assessed by integrating UV-trace at 215 nm (alternatively at 280 nm) and dividing the peak area corresponding to BBI by the sum of the peak areas corresponding to BBI and the impurities and expressing in %. The BBI content (amount) may be determined using either extension coefficient of BBI for the particular wavelength or standard curve of peak areas corresponding to known amounts of BBI.

### Example 3

### Insulin Peptide Degradation by GI Extracts in Presence of BBI

96 well plates were coated by incubating with 0.4% BSA solution for minimum of 60 min. To each well, 210 μl of buffer (HBSS-HEPES buffer with 0.005% Tween 20 and 0.005% BSA, pH 6.5), 30 μl of a substrate (100 μM insulin peptide in buffer) and 30 μl of BBI (1%) were added. The plates were pre-incubated (before adding GI juice) for 60 min at 37° C. After addition of 30 μl of GI juice, the plates were incubated for 60 min/37° C. on shaker. Samples (40 μl) were taken at 0, 3, 6, 10, 30 and 60 min, stopped with 3 vol. cold 96% EtOH (ethanol) w. 1% HCOOH (formic acid) and centrifuged (4500 rcf for 10 min at 4° C.). The resulting supernatant from the samples were diluted 5 times with the buffer prior to LC-MS analysis. Standard samples (0.1, 0.5, 1.0, 5.0, 10.0 μM) were prepared and treated as the samples. Standard curve was analysed both at the beginning and the end of the sequence. Two replicates of each tested conditions were included. Intact insulin peptide was determined in each sample. The results were plotted against the incubation time. Half-lives of the insulin peptide were determined by nonlinear regression of the results using, for example using Graph Pad Prism. The half-lives were expressed relative to the half-live of the insulin without BBI present by dividing the half-lives obtained in the presence of inhibitor with those obtained in the absence of BBI. GI juice was prepared from male Sprague Dawley rats (200-250 g) by excising approximately 20 cm piece of mid jejunum and rinsing the inside with 2.5 ml 0.9% sodium chloride solution. The sodium chloride solution was collected in a centrifuge tube, pooled from all rats (20) and centrifuged at 4500 rpm./10 min/4° C. The supernatant was aliquoted in tubes and stored at −80° C.

The results are listed in table 1.

TABLE 1

| | | *Half Life of | |
|---|---|---|---|
| | | insulin peptide | |
| | *Half Life of | in presence of | *Half Life |
| | insulin peptide | 0.1% BBI | (fold |
| Insulin peptide | (min) | (min) | increase) |
| Insulin 1 | 2.3 ± 0.4 | >400 | >150 |
| Insulin 2 | 1.1 | >1000 | >910 |
| Insulin 3 | 1.2 | 564 | 470 |
| Insulin 4 | 17.9 | >1000 | >56 |
| Insulin 5 | 0.7 | 20 | 29 |
| Insulin 6 | 0.6 | 277 | 462 |
| Insulin 7 | 4.2 | >1000 | >238 |
| Insulin 8 | 2.6 | 630 | 242 |
| Insulin 9 | 43.5 | 100 | 2.3 |
| Insulin(human) | <0.5 | 19 | >38 |

*For experiments repeated more than 2-times, standard deviation is given
Insulin 1: N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[GluA14,HisB25],des-ThrB30-Insulin(human)
Insulin 2: N{A1},N{A1}-dimethyl,N{B1},N{B1}-dimethyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin(human)
Insulin 3: N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-[GluA14,HisB16,HisB25],des-ThrB30-Insulin(human)
Insulin 4: N{Epsilon-B29}-[(4S)-4-carboxy-4-(17-carboxyheptadecanoylamino)butanoyl]-[GluA14,HisB25],des-ThrB27,ThrB30-Insulin(human)
Insulin 5: N{Epsilon-B29}-tetradecanoyl-des-ThrB30-Insulin(human)
Insulin 6: N{Epsilon-B29}-[(4S)-4-carboxy-4-(15-carboxypentadecanoylamino)butanoyl]-des-ThrB30-Insulin(human)
Insulin 7: N{Epsilon-B29}-[(4S)-4-carboxy-4-(15-carboxypentadecanoylamino)butanoyl]-[GluA14,HisB16,HisB25],des-ThrB30-Insulin(human)
Insulin 8: N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[CysA10,GluA14,CysB4,HisB25],des-ThrB30-Insulin(human)
Insulin 9: N{Epsilon-B29}-[(4S)-4-carboxy-4-(19-carboxynonadecanoylamino)butanoyl]-[CysA10,GluA14,CysB3,HisB25],des-ThrB27,ThrB30-Insulin(human)

### Example 4

### GLP-1 Peptide Degradation by GI Extracts in Presence of BBI

96 well plates were coated by incubating with 0.4% BSA solution for a minimum of 60 min. To each well, 210 μl of buffer (HBSS-HEPES buffer with 0.005% Tween 20 and 0.005% BSA, pH 6.5), 30 μl of a substrate (100 μM GLP-1 peptide in buffer) and 30 μl of BBI (1%) were added. The plates were pre-incubated (before adding GI juice) for 60 min at 37° C. After addition of 30 μl of GI juice, the plates were incubated for 60 min/37° C. on shaker. Samples (40 μl) were taken at 0, 3, 6, 10, 30 and 60 min, stopped with 3 vol. cold 96% EtOH w. 1% HCOOH and centrifuged (4500 rcf for 10 min at 4° C.). The resulting supernatant from the samples were diluted 5 times with the buffer prior to LC-MS analysis. Standard samples (0.1, 0.5, 1.0, 5.0, 10.0 μM) were prepared and treated as the samples. Standard curve was analysed both at the beginning and the end of the sequence. Two replicates of each tested conditions were included. Intact GLP-1 peptide was determined in each sample. The results were plotted against the incubation time. Half-lives of the GLP-1 peptide was determined by nonlinear regression of the results using, for example Graph Pad Prism. The half-lives were expressed relative to the half-life of the GLP-1 peptide without BBI present by dividing the half-lives obtained in the presence of inhibitor with those obtained in the absence of BBI. GI juice was prepared from male Sprague Dawley rats (200-250 g) by excising approximately 20 cm piece of mid jejunum and rinsing the inside with 2.5 ml 0.9% sodium chloride solution. The sodium chloride solution was collected in a centrifuge tube, pooled from all rats (20) and centrifuged at 4500 rpm./10 min/4° C. The supernatant was aliquoted in tubes and stored at −80° C.

The results are listed in table 2.

TABLE 2

| | *Half Life of | | |
|---|---|---|---|
| | GLP-1 | | |
| | peptide in | | |
| | presence | *Half Life of | |
| | of 0.1% | GLP-1 | *Half Life |
| | BBI | peptide | (fold |
| GLP-1 peptide | (min) | (min) | increase) |
| N-epsilon26-[2-(2-{2-[2-(2- | 7.6 ± 2.7 | <0.5 | >15 |
| {2-[(S)-4-Carboxy-4-(17- | | | |
| carboxyheptadecanoylamino)- | | | |
| butyrylamino]ethoxy}ethoxy)- | | | |
| acetylamino]ethoxy}ethoxy)ace- | | | |
| tyl]-[Aib8,Arg34]GLP-1-(7-37) | | | |

*For experiments repeated more than 2-times, standard deviation is given

### Example 5

### Transport of Insulin Peptide Across Mucus-Producing E12 Cell Monolayers in the Presence of Sodium Caprate and BBI

### Cell Culturing

HT29-MTX (E12) cells was a gift from David Brayden, University College of Dublin (Dublin, Ireland). Cells were grown in Dulbecco's Modified Eagle Medium supplemented with 10% foetal bovine serum (FBS), 1% penicillin/streptomycin, 1% L-glutamine and 1% non-essential amino acids. For the transport assay, E12 cells were seeded onto tissue culture treated polycarbonate filters in 12-well Transwell plates (1.13 cm², 0.4 μm pore size) at a density of 10⁵ cells/well. Cells were cultured at 37° C. in an atmosphere of 5% CO₂; culture media was exchange every other day. Transport experiments were performed after 14-18 days in culture.

### Transepithelial Transport

The amount of compound transported from the donor chamber (apical side) to the receiver chamber (basolateral side) was measured. The transport study was initiated by adding 400 μl solution (100 μM of insulin peptide+13 mM sodium caprate+/−0.2% Bowman Birk inhibitor (BBI)) and 0.4 μCi/μl [3H]mannitol in transport buffer to the donor chamber and 1000 μl transport buffer to the receiver chamber. The transport buffer consisted of Hank's balanced saline solution containing 10 mM HEPES, 0.1% adjusted to pH 7.4 after addition of compounds. The transport of [3H]mannitol, a marker for paracellular transport, was measured to verify the integrity of the epithelium.

Before the experiment, the E12 cells were equilibrated for 60 min with transport buffer on both sides of the epithelium. Buffer was then removed and the experiment initiated. Donor samples (20 μl) were taken at 0 min and at the end of the experiment. Receiver samples (200 μl) were taken every 15 min. The study was performed in an atmosphere of 5% CO₂-95% O₂ at 37° C. on a shaking plate (30 rpm).

In all samples with insulin peptides and mannitol, the concentration was determined using a LOCI assay and scintillation counter, respectively.

Before and during the experiment the transepithelial electrical resistance (TEER) of the cell monolayers was monitored. In selected experiments, the transport buffer was changed to culturing medium after end of experiment and the TEER measured 24 h after experiment. The TEER was measured with EVOM™ Epithelial Voltohmmeter connected to Chopsticks.

The measured amount of insulin peptide transported from the donor chamber (apical side) to the receiver chamber (basolateral side) for each insulin peptide, sodium caprate, mannitol solution with or without 0.2% BBI is presented in table 3.

TABLE 3

| | Papp | Papp |
|---|---|---|
| Insulin solution | (mean, cm/s) | (SD, cm/s) |
| Insulin 1, sodium caprate | 3.49071E−07 | 4.17027E−08 |
| Insulin 1, sodium caprate, BBI | 5.44653E−07 | 5.15709E−08 |
| Insulin 2, sodium caprate | 3.17755E−07 | 2.45943E−08 |
| Insulin 2, sodium caprate, BBI | 5.28329E−07 | 7.03655E−08 |
| Insulin 3, sodium caprate | 4.99281E−07 | 1.05467E−07 |
| Insulin 3, sodium caprate, BBI | 6.74948E−07 | 9.11499E−08 |
| Insulin 4, sodium caprate | 5.08956E−07 | 3.12406E−08 |
| Insulin 4, sodium caprate, BBI | 9.95926E−07 | 5.51648E−08 |

Papp indicates apparent permeability coefficient

Insulin 1: N{Epsilon-B29}-[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]-[GluA14,HisB25],des-ThrB30-Insulin(human)
Insulin 2: N{A1}, N{A1}-dimethyl, N{B1}, N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin(human)
Insulin 3: N{Epsilon-B29}-[2-[2-[2-[[2-[2-[2-[[(4S)-4-carboxy-4-(19-carboxynonadecanoyl-amino)butanoyl]amino]ethoxy]ethoxy]acetyl]amino]ethoxy]ethoxy]acetyl]-[GluA14,HisB16,HisB25],des-ThrB30-Insulin(human)
Insulin 4: N{Epsilon-B29}-[(4S)-4-carboxy-4-(17-carboxyheptadecanoylamino)butanoyl]-[GluA14,HisB25],des-ThrB27,ThrB30-Insulin(human)

### Example 6

### Transport of GLP-1 Peptide Across Mucus-Producing E12 Cell Monolayers in the Presence of Sodium Caprate and BBI

### Cell Culturing

HT29-MTX (E12) cells was a gift from David Brayden, University College of Dublin (Dublin, Ireland). Cells were grown in Dulbecco's Modified Eagle Medium supplemented with 10% foetal bovine serum (FBS), 1% penicillin/streptomycin, 1% L-glutamine and 1% non-essential amino acids. For the transport assay, E12 cells were seeded onto tissue culture treated polycarbonate filters in 12-well Transwell plates (1.13 cm², 0.4 μm pore size) at a density of 10⁵ cells/well. Cells were cultured at 37° C. in an atmosphere of 5% CO₂; culture media was exchange every other day. Transport experiments were performed after 14-18 days in culture.

### Transepithelial Transport

The amount of GLP-1 peptide transported from the donor chamber (apical side) to the receiver chamber (basolateral side) was measured. The transport study was initiated by adding 400 μl solution (100 μM of GLP-1 peptide+13 mM sodium caprate+/−0.2% Bowman Birk inhibitor (BBI)) and 0.4 μCi/μl [3H]mannitol in transport buffer to the donor chamber and 1000 μl transport buffer to the receiver chamber. The transport buffer consisted of Hank's balanced saline solution containing 10 mM HEPES, 0.1% adjusted to pH 7.4 after addition of compounds. The transport of [3H]mannitol, a marker for paracellular transport, was measured to verify the integrity of the epithelium.

Before the experiment, the E12 cells were equilibrated for 60 min with transport buffer on both sides of the epithelium. Buffer was then removed and the experiment initiated. Donor samples (20 μl) were taken at 0 min and at the end of the experiment. Receiver samples (200 μl) were taken every 15 min. The study was performed in an atmosphere of 5% CO₂-95% O₂ at 37° C. on a shaking plate (30 rpm).

In all samples with GLP-1 peptide and mannitol, the concentration of GLP-1 peptide was determined using a LOCI assay (or LC-MS assay) and scintillation counter, respectively.

Before and during the experiment the transepithelial electrical resistance (TEER) of the cell monolayers was monitored. In selected experiments, the transport buffer was changed to culturing medium after end of experiment and the TEER measured 24 h after experiment. The TEER was measured with EVOM™ Epithelial Voltohmmeter connected to Chopsticks.

The measured amount of GLP-1 peptide transported from the donor chamber (apical side) to the receiver chamber (basolateral side) for each GLP-1 peptide, sodium caprate, mannitol solution with or without 0.2% BBI is presented in table 4.

TABLE 4

| | Papp | Papp |
|---|---|---|
| GLP-1 solution | (mean, cm/s) | (SD, cm/s) |
| GLP-1 peptide 1, sodium caprate | 1.48132E−07 | 1.66844E−08 |
| GLP-1 peptide 1, sodium caprate, | 3.19278E−07 | 5.74768E−08 |
| 0.2% BBI | | |
| GLP-1 peptide 2, sodium caprate | 3.89E−07 | 2.18213E−08 |
| GLP-1 peptide 2, sodium caprate, | 4.38299E−07 | 5.85288E−08 |
| 0.2% BBI | | |
| GLP-1 peptide 3, sodium caprate | 9.0672E−08 | 4.32679E−09 |
| GLP-1 peptide 3, sodium caprate, | 1.25404E−07 | 2.13832E−08 |
| 0.2% BBI | | |
| GLP-1 peptide 4, sodium caprate | 2.03658E−07 | 1.66398E−08 |
| GLP-1 peptide 4, sodium caprate, | 5.28788E−07 | 8.62404E−08 |
| 0.2% BBI | | |
| GLP-1 peptide 5, sodium caprate | 3.1387E−07 | 6.26555E−08 |
| GLP-1 peptide 5, sodium caprate, | 5.91753E−07 | 3.77236E−08 |
| 0.2% BBI | | |
| GLP-1 peptide 6, sodium caprate | 1.58934E−07 | 1.0132E−08 |
| GLP-1 peptide 6, sodium caprate, | 2.95624E−07 | 2.58552E−08 |
| 0.2% BBI | | |
| GLP-1 peptide 7, sodium caprate | 1.08637E−07 | 2.00018E−08 |
| GLP-1 peptide 7, sodium caprate, | 1.81771E−07 | 6.87461 E−08 |
| 0.2% BBI | | |
| GLP-1 peptide 8, sodium caprate | 1.83585E−07 | 2.11939E−08 |
| GLP-1 peptide 8, sodium caprate, | 3.62972E−07 | 4.2416E−08 |
| 0.2% BBI | | |
| GLP-1 peptide 9, sodium caprate | 2.72E−07 | 2.28E−08 |
| GLP-1 peptide 9, sodium caprate, | 3.79E−07 | 8.46E−08 |
| 0.2% BBI | | |
| GLP-1 peptide 10, sodium caprate | 2.76E−07 | 3.49E−08 |
| GLP-1 peptide 10, sodium caprate, | 5.10E−07 | 1.45E−08 |
| 0.2% BBI | | |

Papp indicates apparent permeability coefficient

GLP-1 peptide 1: N{Epsilon-26}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[Aib8,Arg34]-GLP-1-(7-37)-peptide
GLP-1 peptide 2: N{Epsilon-18}-[2-[2-[2-[[2-[2-[2-[[(4S)-4-carboxy-4-[10-(4-carboxyphenoxy)-decanoylamino]butanoyl]amino]ethoxy]ethoxy]acetyl]amino]ethoxy]ethoxy]acetyl], N{Epsilon-26}-[2-[2-[2-[[2-[2-[2-[[(4S)-4-carboxy-4-[10-(4-carboxyphenoxy)decanoylamino]butanoyl]-amino]ethoxy]ethoxy]acetyl]amino]ethoxy]ethoxy]acetyl]-[Aib8,Lys18,Glu22,Gln34]-GLP-1-(7-37)-peptide
GLP-1 peptide 3: N{Epsilon-26}-[(2S)-2-amino-6-[[(2S)-2-amino-6-[[(4S)-4-carboxy-4-(15-carboxypentadecanoylamino)butanoyl]amino]hexanoyl]amino]hexanoyl], N{Epsilon-37}-[(2S)-2-amino-6-[[(2S)-2-amino-6-[[(4S)-4-carboxy-4-(15-carboxypentadecanoylamino)butanoyl]-amino]hexanoyl]amino]hexanoyl]-[Aib8,Arg34,Lys37]-GLP-1-(7-37)-peptide
GLP-1 peptide 4: N{Epsilon-18}-[2-[2-[2-[[2-[2-[2-[[(4S)-4-carboxy-4-[10-(4-carboxyphenoxy)-decanoylamino]butanoyl]amino]ethoxy]ethoxy]acetyl]amino]ethoxy]ethoxy]acetyl], N{Epsilon-26}-[2-[2-[2-[[2-[2-[2-[[(4S)-4-carboxy-4-[10-(4-carboxyphenoxy)decanoylamino]butanoyl]-amino]ethoxy]ethoxy]acetyl]amino]ethoxy]ethoxy]acetyl]-[Aib8,Lys18,Glu22,Arg34]-GLP-1-(7-37)-peptide
GLP-1 peptide 5: N{Epsilon-18}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl], —N{Epsilon-26}-[2-[2-[2-[[2-[2-[2-[[(4S)-4-carboxy-4-(13-carboxytridecanoylamino)butanoyl]-amino]ethoxy]ethoxy]acetyl]amino]ethoxy]ethoxy]acetyl]-[Aib8,Lys18,Glu22,Arg34]-GLP-1-(7-37)-peptide
GLP-1 peptide 6: N{Epsilon-18}-[2-[2-[2-[[2-[2-[2-[[(4S)-4-carboxy-4-[10-(4-carboxyphenoxy)-decanoylamino]butanoyl]amino]ethoxy]ethoxy]acetyl]amino]ethoxy]ethoxy]acetyl], N{Epsilon-26}-[2-[2-[2-[[2-[2-[2-[[(4S)-4-carboxy-4-[10-(4-carboxyphenoxy)decanoylamino]butanoyl]-amino]ethoxy]ethoxy]acetyl]amino]ethoxy]ethoxy]acetyl]-[Aib8,Lys18,Gln34]-GLP-1-(7-37)-peptide
GLP-1 peptide 7: N{Epsilon-26}-[2-[2-[2-[[2-[2-[2-[[(4S)-4-carboxy-4-(15-carboxy-pentadecanoylamino)butanoyl]amino]ethoxy]ethoxy]acetyl]amino]ethoxy]ethoxy]acetyl]-[Aib8,Arg34]-GLP-1-(7-37)-peptide
GLP-1 peptide 8: N{Epsilon-26}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[Aib8,His31,Gln34]-GLP-1-(7-37)-peptide
GLP-1 peptide 9: N{Epsilon-26}-[2-[2-[2-[[2-[2-[2-[[(4S)-4-carboxy-4-[10-(4-carboxyphenoxy)-decanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[10-(4-carboxyphenoxy)decanoylamino]butanoyl]-amino]ethoxy]ethoxy]acetyl]amino]ethoxy]ethoxy]acetyl]-[Aib8,Arg34, Lys37]-GLP-1-(7-37)-peptide
GLP-1 peptide 10: N{Epsilon-26}-[2-[2-[2-[[(4S)-4-carboxy-4-[[2-[2-[2-(13-carboxytridecanoylamino)ethoxy]ethoxy]acetyl]amino]butanoyl]amino]ethoxy]ethoxy]acetyl], N {Epsilon-37}-[2-[2-[2-[[(4S)-4-carboxy-4-[[2-[2-[2-(13-carboxytridecanoylamino)ethoxy]-ethoxy]acetyl]amino]butanoyl]amino]ethoxy]ethoxy]acetyl]-[Aib8,Arg34, Lys37]-GLP-1-(7-37)-peptide

### Example 7

### Preparation of BBI for Solid Formulation

Dry purified BBI was dissolved in water to a concentration of 20 mg/ml at an adjusted pH of 8.0. The solution was spray-dried on a Büchi Mini-Spray Dryer B-290 equipped with a small diameter cyclone and a 0.7 mm/1.5 mm nozzle. The process conditions were: air inlet temperature: 120° C.; solution flow rate: 4 ml/min solution flow; nitrogen flow rate: 35 mm, aspirator: 100%. The spray-dried powder was used as is in the solid formulations.

### Example 8

### Hard Capsules Comprising Insulin Peptide, Sodium Caprate (Sodium Decanoate) and BBI

Formulation of a hard capsule dry filling material according to the present invention was performed as outlined here, this example concerns formulations of the present invention comprising:

Insulin peptide 1.39% (w/w)

BBI 8.33% (w/w)

Sodium decanoate (Sodium caprate) 45.83% (w/w)

Sorbitol 43.93% (w/w)

Magnesium stearate 0.52% (w/w)

The production procedure for dry filling of 18 hard-shelled capsules was performed as follows:

The milled insulin peptide powder was passed through a sieve with a 0.25 mm mesh size. After sieving, the correct amount of insulin peptide was weighed. Sorbitol powder was passed through a sieve with a 0.5 mm mesh size. After sieving the correct sorbitol amount was weighed. Insulin peptide and sorbitol were mixed in a small container. An amount of sorbitol equivalent to the amount of insulin peptide was added to said container and stirred by hand. Then the double amount of sorbitol relative to the previous addition was added and stirred by hand until insulin peptide and all sorbitol were mixed well. This was followed by mechanical mixing in a Turbula-mixer to finalize the mixing to obtain a homogeneous powder. Sodium caprate (in the form of roller compacted granulate) was then added to the insulin peptide-sorbitol powder according to equal volumes principle and finalized with a mechanical mixing step in a Turbula-mixer. Finally magnesium stearate was passed through a sieve with a 0.25 mm mesh size. Magnesium stearate was weighed and added to the powder and mixed mechanically in a Turbula-mixer.

The powder was then filled into HPMC hard capsules, size 00 (Quali-V, Qualicaps), to a fill weight of 600 mg/capsule.

When capsules with a higher concentration of the BBI (such as 33%) were manufactured the increased volume of BBI was compensated in correspondence by reducing the overall amount of sorbitol, see Table 5.

When capsules with a lower concentration of the BBI (such as 4.2%) were manufactured the decreased volume of BBI was compensated in correspondence by increasing the overall amount of sorbitol, see Table 5.

When capsules with a lower concentration of the BBI (such as 1.7%) were manufactured the decreased volume of BBI was compensated in correspondence by increasing the overall amount of sorbitol, see Table 5.

TABLE 5
Formulation of capsule fill material with various BBI ratios

| Concentration of BBI | 1.7% | | 4.2% | | 33% | |
|---|---|---|---|---|---|---|
| Insulin peptide | 1.39% | 0.12 g | 1.39% | 0.13 g | 1.39% | 0.12 g |
| BBI | 1.67% | 0.15 g | 4.17% | 0.39 g | 33.3% | 3.00 g |
| Sodium caprate | 45.83% | 4.13 g | 45.83% | 4.26 g | 45.83% | 4.13 g |
| Sorbitol | 50.59% | 4.56 g | 48.10% | 4.47 g | 18.92% | 1.71 g |
| Magnesium stearate | 0.52% | 0.05 g | 0.52% | 0.05 g | 0.52% | 0.05 g |
| Total | 100% | 9.00 g | 100% | 9.30 g | 100% | 9.00 g |

When capsules with a higher concentration of the sodium caprate content (such as 58%) were manufactured the increased volume of capric acid salt was compensated in correspondence by reducing the overall amount of sorbitol, see Table 6.

When capsules with a higher concentration of the sodium caprate content (such as 75%) were manufactured the increased volume of capric acid salt was compensated in correspondence by reducing the overall amount of sorbitol, see Table 6.

When capsules with a lower concentration of the sodium caprate content (such as 25%) were manufactured the decreased volume of capric acid salt was compensated in correspondence by increasing the amount of sorbitol added, see Table 6.

TABLE 6
Formulation of capsule fill material with various capric acid salt ratios

| | Concentration of Sodium caprate | | | | | |
|---|---|---|---|---|---|---|
| | 25% | | 58% | | 75% | |
| Insulin peptide | 1.39% | 0.13 g | 1.39% | 0.13 g | 1.39% | 0.12 g |
| BBI | 8.33% | 0.78 g | 8.33% | 0.78 g | 8.33% | 0.73 g |
| Sodium caprate | 25.00% | 2.33 g | 58.33% | 5.43 g | 75.00% | 6.53 g |
| Sorbitol | 64.76% | 6.02 g | 31.43% | 2.92 g | 14.76% | 1.29 g |
| Magnesium stearate | 0.52% | 0.05 g | 0.52% | 0.05 g | 0.52% | 0.05 g |
| Total | 100% | 9.3 g | 100% | 9.3 g | 100% | 8.7 g |

When capsules with a lower concentration of the BBI (such as 1.7%) and a higher concentration of the sodium caprate content (such as 75%) were manufactured the difference in volume of the combined BBI and sodium caprate was compensated in correspondence by decreasing the overall amount of sorbitol, see Table 7.

TABLE 7
Formulation of capsule fill material with
various BBI and capric acid salt ratios

| Concentration of BBI | 1.7% |
|---|---|
| Concentration of | 75% |
| Sodium caprate | |

| | | |
|---|---|---|
| Insulin peptide | 1.39% | 0.12 g |
| BBI | 1.67% | 0.15 g |
| Sodium caprate | 75.00% | 6.53 g |
| Sorbitol | 21.43% | 1.86 g |
| Magnesium stearate | 0.52% | 0.05 g |
| Total | 100% | 8.7 g |

Hard-shelled capsules prepared as described in this example were coated with an enteric coating.

For this purpose polymers of the copolymer family denominated “Poly(methacrylic acid-co-ethyl acrylate)” (Brand name Eudragit L30D55®) were used. 105.0 g of an aqueous dispersion Poly(methacrylic acid-co-ethyl acrylate) (Brand name Eudragit L30D55®) was placed in a beaker on a suitable stirring apparatus. Glycerol monostearate, plasticizing agent triethyl citrate and polyoxyethylene (20) sorbitan monooleate in the form of 15.8 g PlasAcryl T20®, 4.8 g triethyl citrate, 1.8 g Tween 80 and 72.6 g pure water were added to the amount of 15.75% of the total dry polymer. The ingredients were added to said aqueous emulsion of poly(methacrylic acid-co-ethyl acrylate) (Brand name Eudragit L30D55®). The mixture was allowed to mix for 60 minutes prior to a filtration through a 0.24 mm mesh filter to remove lumps.

The coating of the hard capsules was performed in a pan coater or fluid bed coater. In a pan coater with the pan size of 8.5″, with a conventional patterned air Schlick spray nozzle with an orifice of 1.0 mm, an atomizing and pattern air pressure of 0.5-0.6 bar, inlet air temperature of 35° C., air flow of 130 kg/hours, the coating was performed by pumping the polymer solution in through the nozzle. After addition of 5-7% w/w polymer distributed evenly on the hard capsules, the spraying was stopped.

### Example 9

### Hard Capsules Comprising GLP-1 Peptide, Sodium Caprate (Sodium Decanoate) and BBI

Formulation of a hard capsule dry filling material according to the present invention was performed as outlined here, this example concerns formulations of the present invention comprising:

| | |
|---|---|
| GLP-1 peptide | 3.39% (w/w) |
| BBI | 8.33% (w/w) |
| Sodium decanoate (Sodium caprate) | 45.83% (w/w) |
| Sorbitol | 41.93% (w/w) |
| Magnesium stearate | 0.52% (w/w) |

The procedure was performed as follows:

The correct amount of spray-dried GLP-1 peptide was weighed. Sorbitol powder was passed through a sieve with a 0.5 mm mesh size. After sieving the correct amount was weighed. In a small container GLP-1 peptide and sorbitol were mixed. An amount of sorbitol equivalent to the amount of GLP-1 peptide was added to said container and stirred by hand. Then the double amount of sorbitol relative to the previous addition was added and stirred by hand until insulin and all sorbitol were mixed well. This was followed by a mechanical mixing in a Turbula-mixer to finalize the mixing to obtain a homogeneous powder. Sodium caprate (in the form of roller compacted granulate) was then added to the GLP-1 peptide-sorbitol powder according to equal volumes principle. This was done in two steps and finalized with a mechanical mixing step in a Turbula-mixer. Finally magnesium stearate was passed through a sieve with 0.25 mm mesh size. Magnesium stearate was weighed and added to the powder and mixed mechanically. The powder was then filled into HPMC hard capsules, size 00 (Quali-V, Qualicaps), to a fill weight of 600 mg/capsule.

When capsules with a higher concentration of the BBI (such as 33%) were manufactured the increased volume of BBI was compensated in correspondence by reducing the overall amount of sorbitol, see Table 8.

When capsules with a lower concentration of the BBI (such as 1.7%) were manufactured the decreased volume of BBI was compensated in correspondence by increasing the overall amount of sorbitol, see Table 8.

TABLE 8
Formulation of capsule fill material with various BBI ratios

| Concentration of BBI | 1.7% | | 33% | |
|---|---|---|---|---|
| GLP-1 peptide | 3.34% | 0.30 g | 3.34% | 0.30 g |
| BBI | 1.67% | 0.15 g | 33.33% | 3.00 g |
| Sodium caprate | 45.83% | 4.13 g | 45.83% | 4.13 g |
| Sorbitol | 48.64% | 4.38 g | 16.97% | 1.53 g |
| Magnesium stearate | 0.52% | 0.05 g | 0.52% | 0.05 g |
| Total | 100% | 9.00 g | 100% | 9.00 g |

### Example 10

### Dog PD/PK results for insulin peptide N{Epsilon-B29}-[2-[2-[2-[[2-[2-[2-[[(4S)-4-carboxy-4-(17-carboxyheptadecanoylamino)butanoyl]amino]ethoxy]ethoxy]-acetyl]amino]ethoxy]ethoxy]acetyl]-[GluA14,HisB25],des-ThrB30-Insulin(human)

Preparation of capsules is described in Example 8. Each capsule contained 8 mg of the insulin peptide N{Epsilon-B29}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[GluA14,HisB25],des-ThrB30-Insulin(human). Each study was performed in 8 male Beagle dogs (HsdRcc:DOBE) from Harlan Gannat, France, about 36-48 weeks old and in the weight range of about 9-11 kg at arrival. The animals were dosed orally with coated capsules. The capsules were administered immediately after the t=0 min sample was drawn. The capsule was placed in the back of the mouth of the dog, so the dog swallowed the capsule without chewing it. After the capsule was swallowed, 10 ml water was administered into the mouth by a syringe.

A full plasma concentration-time profile was obtained for each animal.

### Blood Samples:

For each time point, the first drops of blood were discarded. Approx. 800 μl blood was collected in 1.5 ml EDTA eppendorf tubes for plasma and a 10 ul capillary tube was filled for glucose:

### Time Points:

Predose (0), and 15, 30, 45, 60, 75, 90, 105, 120, 135, 150, 165, 180, 210, 240, 270, 300, 360, 480, 600, 720 minutes, 24, 30, 48 and 72 hours.

During periods of frequent sampling, the blood samples were taken from Venflon catheters in cephalic veins kept open with heparin saline. The other blood samples were taken from a jugular vein.

Blood samples were kept on wet ice for max 20 min. before centrifugation at 4° C., 4 min., 1300 G.

Plasma was immediately transferred to two micronic tubes, 80 μl plasma in each, from each blood sample and stored at −20° C. until assayed.

The glucose concentration was analysed by the immobilised glucose oxidase method using 10 μl whole blood immersed into 500 μl analysis buffer (Biosen auto-analyser and buffer solution).

### Analysis of Plasma Samples:

The plasma was analysed for insulin peptide using a Luminescence Oxygen Channeling Immunoassay (LOCI). The LOCI assay employed donor beads coated with streptavidin and acceptor beads conjugated with a monoclonal antibody binding to a mid-molecular region of insulin peptide. The other monoclonal antibody, specific for an N-terminal epitope, was biotinylated. In the assay the three reactants were combined with the insulin peptide which form a two-sited immuno-complex. Illumination of the complex released singlet oxygen atoms from the donor beads which channels into the acceptor beads and trigger chemiluminescence which was measured in the EnVision plate reader. The amount of light is proportional to the concentration of insulin peptide and the lower limit of quantification (LLOQ) in plasma is 100 pM.

Plasma concentration-time profiles were analysed by a non-compartmental pharmacokinetics analysis in WinNonlin 5.2 (Pharsight Inc., Mountain View, Calif., USA). Calculations were performed using individual concentration-time values from each animal. For the calculations of oral bioavailability iv data from previous studies in Beagle dogs were applied.

The results are summarized in Table 9.

TABLE 9
Insulin bioavailability after oral dosing to dogs in capsules
containing a BBI solubilizing agent, sodium caprate and BBI

| | | | BBI | | |
|---|---|---|---|---|---|
| Sodium | | BBI | solubilizing | Bioavail- | Standard |
| caprate | BBI | solubilizing | agent | ability | deviation |
| (mg) | (mg) | agent | (mg) | (%) | (%) |
| 150 | 0 | Sorbitol | 439 | 1.3 | 1.5 |
| 150 | 50 | Sorbitol | 389 | 1.7 | 2.1 |
| 275 | 0 | Sorbitol | 325 | 1.8 | 2.1 |
| 275 | 10 | Sorbitol | 315 | 2.4 | 2.0 |
| 275 | 25 | Sorbitol | 300 | 4.2 | 3.7 |
| 275 | 50 | Sorbitol | 275 | 4.8 | 3.9 |
| 275 | 50 | Sorbitol | 275 | 7.1 | 5.8 |
| | | | | (N = 32) | |
| 275 | 200 | Sorbitol | 125 | 3.0 | 2.6 |
| 275 | 50 | Microcrystaline | 214 | 2.9 | 1.4 |
| | | cellulose - | | | |
| | | insoluble filler | | | |
| 275 | 50 | No BBI | 0 | 2.5 | 1.9 |
| | | solubilizing | | | |
| | | agent | | | |
| 350 | 0 | Sorbitol | 250 | 2.5 | 2.8 |
| 350 | 50 | Sorbitol | 200 | 7.2 | 5.0 |
| 450 | 0 | Sorbitol | 150 | 2.8 | 3.6 |
| | | | | (N = 16) | |
| 450 | 10 | Sorbitol | 140 | 5.1 | 6.0 |
| 450 | 50 | Sorbitol | 100 | 5.7 | 5.0 |

### Example 11

### Dog PD/PK Results for GLP-1 Peptides

Preparation of capsules is described in Example 9. Each capsule contained 20 mg of the GLP-1 peptide N{Epsilon-26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Aib8,Arg34]-GLP-1-(7-37)-peptide. Each study was performed in 8 male Beagle dogs.

The animals were either dosed intraduodenally with uncoated capsules using an endoscope or dosed PO with coated capsules (coated in a similar manner as described for insulin capsules in example 8). For the endoscopic procedure the animals were sedated with medetomidin i.v. and propofol i.v. The capsules were dosed to duodenum using endoscope equipped with a custom-made device that ensured capsules can be delivered to duodenum without being exposed to liquid in the oesophagus and stomach. The dogs were dosed with an antidote to the sedation (antisedan) that counteracted the medetomidin and the iv propofol was cleared fast. The dogs were awake 15-30 min after dosing. For the PO procedure the capsule was placed in the back of the mouth of the dog, so the dog swallowed the capsule without chewing it. 10 mL of water was provided into the mouth with a syringe to facilitate swallowing of the capsule. For the PO studies with coated capsules gastric acid secretion was induced before administration of the oral tablet. Pentagastrin was administered subcutaneously at a dose of 4 μg/kg body weight (120 μg/mL) was administered 20 minutes prior to the PO dose.

In a standard blood sampling regimen 20 samples (pre-dose to 11 days) were used to obtain a full plasma concentration-time profile for each animal.

### Blood Samples:

For each time point, the first drops of blood were discarded. Approx. 800 μl blood was collected in 1.5 ml EDTA eppendorf tubes for plasma and a 10 ul capillary tube was filled for glucose:

### Time Points:

Predose, 0.25, 0.5, 0.75, 1, 1.5, 2, 2.5, 3, 4, 6, 8, 10, 24, 48, 72, 96, 120, 144, 168, 192, 216, 240, 264, and 288 h hours post dosing.

Blood samples were kept on wet ice for max 20 min. before centrifugation at 4° C., 4 min., 1300 G. Plasma was immediately transferred to two micronic tubes, 80 μl plasma in each, from each blood sample and stored at −20° C. until assayed.

The glucose concentration was analysed by the immobilised glucose oxidase method using 10 μl whole blood immersed into 500 μl analysis buffer (Biosen auto-analyser and buffer solution).

### Analysis of Plasma Samples:

Plasma was analysed for GLP-1 peptide using a Luminescence Oxygen Channeling Immunoassay (LOCI). The LOCI assay employed donor beads coated with streptavidin and acceptor beads conjugated with a monoclonal antibody binding to a mid-molecular region of GLP-1 peptide. The other monoclonal antibody, specific for an N-terminal epitope, was biotinylated. In the assay the three reactants were combined with the GLP-1 peptide to form a two-sited immuno-complex. Illumination of the complex released singlet oxygen atoms from the donor beads which channels into the acceptor beads and trigger chemiluminescence which was measured in the EnVision plate reader. The amount of light was proportional to the concentration of GLP-1 peptide and the lower limit of quantification (LLOQ) in plasma was 100 pM.

Plasma concentration-time profiles were analysed by a non-compartmental pharmacokinetics analysis in Phoenix WinNonlin 6.3 (Pharsight Inc., Mountain View, Calif., USA). Calculations were performed using individual concentration-time values from each animal. For the calculations of oral bioavailability iv data from previous studies in Beagle dogs were applied.

The results are summarized in Table 10.

TABLE 10
GLP-1 peptide bioavailability after endoscope dosing to dogs in capsules
containing a BBI solubilizing agent, sodium caprate and BBI

| | | | BBI | |
|---|---|---|---|---|
| Sodium | | BBI | solubilizing | Bioavail- |
| caprate | BBI | solubilizing | agent | ability |
| (mg) | (mg) | agent | (mg) | (%) |
| 275 | 0 | Sorbitol | 325 | 0.10 |
| 275 | 10 | Sorbitol | 315 | 0.38 |
| 275 | 50 | Sorbitol | 275 | 0.31 |
| 275 | 200 | Sorbitol | 125 | 0.18 |

TABLE 11
GLP-1 peptide bioavailability after PO dosing to dogs in capsules
containing a BBI solubilizing agent, sodium caprate and BBI

| | | | BBI | |
|---|---|---|---|---|
| Sodium | | BBI | solubilizing | Bioavail- |
| caprate | BBI | solubilizing | agent | ability |
| (mg) | (mg) | agent | (mg) | (%) |
| 275 | 0 | Sorbitol | 300 | 0.04 |
| 275 | 50 | Sorbitol | 250 | 0.28 |
| 450 | 0 | Sorbitol | 125 | 0.27 |
| 450 | 50 | Sorbitol | 75 | 0.57 |

### Example 12

### Preparation of Insulin Tablet

Formulation of Tablet Core Material, Compressing Tablet Core and Coating Tablet Comprising Insulin, Bowman-Birk Inhibitor and Sodium Salt of Capric Acid (Sodium Decanoate)

Formulation of a tablet core material was performed as outlined here, this example concerns formulations of the present invention comprising:

| | |
|---|---|
| Insulin | 1.09% (w/w) |
| Sodium decanoate (Sodium salt of capric acid) | 72.37% (w/w) |
| Sorbitol | 19.49% (w/w) |
| Bowman-Birk inhibitor | 6.58% (w/w) |
| Stearic acid | 0.47% (w/w) |

The procedure was performed as follows:

Insulin powder was sieved using a sieve of mesh size 0.3 mm. After sieving the correct amount of insulin was weighed. Sorbitol powder was sieved using a sieve of mesh size 0.5 mm. After sieving the correct amount was weighed.

In a small container insulin and sorbitol were mixed. An amount of sorbitol equivalent to the amount of insulin was added to said container and stirred by hand. Then the double amount of sorbitol relative to the previous addition was added and stirred by hand until insulin and all sorbitol were mixed well. This was followed by a mechanical mixing in a Turbula-mixer to finalize the mixing to obtain a homogeneous powder. The Bowman-Birk inhibitor was added to the mixture and mixed manually.

Sodium salt of capric acid (in the form of roller compacted granulate) was then added to the insulin-sorbitol powder according to equal volumes principle. This was done in two steps and finalized with a mechanical mixing step in a Turbula-mixer.

Finally stearic acid was sieved using a sieve of mesh size 0.3 mm. Stearic acid was weighed and added to the powder and mixed mechanically.

The powder was then compressed in a rotary tablet press to form tablets of a mass of 760 mg.

The tablet core described above was coated with a polymer enteric coating.

In a pan coater with the pan size of 8.5″, with a conventional patterned air Schlick spray nozzle with an orifice of 1.0 mm, an atomizing and pattern air pressure of 0.55 bar, inlet air temperature of 36° C., air flow of 100 m3/hour, the coating was performed by pumping the polymer solution in through the nozzle. After addition of 7-8% w/w polymer distributed evenly on the tablet cores, the coating was stopped.

After finalized application of the enteric coating the product was kept in the coating equipment at a low rotational speed for 5 minutes to allow for cooling below 28° C. to avoid intertabletal sticking.

After removal of the product from the coating equipment the product was exposed to the elevated temperature in a heating cabinet at 40° C. for 2 hours in order to enable the polymer curing.

### Example 13

### Dog PD/PK results for insulin peptide N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]-amino]ethoxy]ethoxy]acetyl]-[GluA14,HisB25],des-ThrB30-Insulin(human) in tablet

| Sodium | | | |
|---|---|---|---|
| caprate | BBI | Sorbitol | F |
| (mg) | (mg) | (mg) | (%) |
| 550 | 0 | 150 | 6.9 |
| 550 | 50 | 150 | 7.7 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A solid oral pharmaceutical composition comprising
a) an active peptide ingredient which is an insulin peptide or GLP-1 peptide,
b) a salt of capric acid,
c) Bowman-Birk Inhibitor (BBI), and
d) a BBI solubilizing agent.

2. The solid oral pharmaceutical composition of claim 1, wherein at least 10 mg salt of capric acid and at least 1 mg BBI are present per dosage form.

3. The solid oral pharmaceutical composition of claim 1, comprising 10 mg-450 mg salt of capric acid and 1 mg-200 mg BBI.

4. The solid oral pharmaceutical composition of claim 1, comprising 10 mg-400 mg BBI solubilizing agent.

5. The solid oral pharmaceutical composition of claim 1, wherein the solid oral pharmaceutical composition is in the form of a powder or granulate comprised in a capsule.

6. The solid oral pharmaceutical composition of claim 5, wherein the capsule comprises up to 1000 mg powder.

7. The solid oral pharmaceutical composition of claim 5, wherein the capsule is a hard capsule.

8. The solid oral pharmaceutical composition of claim 1, wherein BBI is isolated from plants.

9. The solid oral pharmaceutical composition of claim 1, wherein the purity of BBI is at least 90% pure.

10. The solid oral pharmaceutical composition of claim 1, wherein the insulin peptide or the GLP-1 peptide is acylated.

11. The solid oral pharmaceutical composition of claim 1, wherein the active peptide ingredient is an insulin peptide which is acylated.

12. The solid oral pharmaceutical composition of claim 1, wherein the active peptide ingredient is a GLP-1 peptide which is acylated.

13. The solid oral pharmaceutical composition of claim 1, wherein the ratio (w/w) of the salt of capric acid to BBI is 45:1 or less.

14. The solid oral pharmaceutical composition of claim 1, wherein the ratio (w/w) of the salt of capric acid to BBI is 5.5:1 or less.

15. The solid oral pharmaceutical composition of claim 1, wherein the ratio (w/w) of the salt of capric acid to BBI is about 1:1.

16. The solid oral pharmaceutical composition of claim 1, wherein the salt of capric acid is sodium caprate.

17. The solid oral pharmaceutical composition of claim 1, wherein the BBI solubilizing agent is a sugar alcohol.

18. The solid oral pharmaceutical composition of claim 1, wherein the BBI solubilizing agent is sorbitol.

19. The solid oral pharmaceutical composition of claim 6, wherein the capsule is a hard capsule.


SEQUENCE LISTING (3 sequences)

SEQ ID NO: 1 - protein, 31 aa, Artificial
  Modified GLP-1(7-37)
HXEGTFTSDVSSYLEGQAAKEFIAWLVRGRG

SEQ ID NO: 2 - protein, 21 aa, Artificial
  Modified insulin A chain
GIVEQCCTSICSLEQLENYCN

SEQ ID NO: 3 - protein, 29 aa, Artificial
  Modified insulin B chain
FVNQHLCGSHLVEALYLVCGERGFHYTPK